(19) United States
(12) Patent Application Publication
(10) Pub. No.: US 2004/0067998 A1
(43) Pub. Date: Apr. 8, 2004

(54) Antifungal and/or antiparasitic pharmaceutical composition and novel indole derivatives as active principle of such a composition

(76) Inventors: Marc Le Borgne, Nantes (FR); Young Min Na, Yusong-gu Daejeon (KR); Fabrice Pagniez, Nantes (FR); Guillaume Le Baut, Saint Sebastien sur Loire (FR); Patrice Le Pape, Vertou (FR); Hiam Abdala, Nantes (FR)

Correspondence Address: FULBRIGHT & JAWORSKI L.L.P., 600 CONGRESS AVE., SUITE 2400, AUSTIN, TX, 78701, US

(21) Appl. No.: 10/381,293

(22) PCT Filed: Sep. 21, 2001
(86) PCT No.: PCT/FR01/02950

(51) Int. Cl. A61K 31/4178; C 07D 4 3/04

(52) U.S. Cl. 514/397; 548/312.1

(57) ABSTRACT

The present invention relates to novel indole derivatives, their method of preparation and their pharmacological activity as antimycotic and/or antiparasitic compounds.

The present invention relates to novel indole derivatives, their method of preparation and their pharmacological activity as antimycotic and/or antiparasitic compounds.

Antifungal compositions in general have been described by Joly et al. (1994), Andriole et al. (1999) and by Georgopapadakou (1996).

Variously substituted (1H-imidazol-ylmethyl)indoles, disclosed in the U.S. Pat. No. 4,410,539, have been proposed as inhibitors of thromboxane synthetase. In addition azolylmethylbenzimidazoles and benzotriazoles are claimed as having anti-androgen (EP 0 260 744 A2) or anti-estrogen activities (EP 0 293 978 A2).

Some compounds derived from azolylmethyl- or azolylbenzyl-indoles have been described as inhibitors of P450 aromatase, able to be used in the treatment of hormonal problems, particularly in the treatment of hormonal disorders associated with menopause or in the treatment of prostate cancers (Le Borgne et al., 1997; Le Borgne et al., 1999; Marchand et al., 1998).

Compounds of the type 1-benzyl-3-(1-imidazolylmethyl)indoles or 1-ethyl-3-[α-(1-imidazolyl)phenethyl]-indoles have been described as having pharmacological properties against micro-organisms such as Candida or Cryptococcus neoformans (Gatti R. et al., 1985; Cavrini M. et al., 1984).

The applicant has shown according to the invention that new compounds derived from indole have antifungal and antiparasitic properties and thus constitute useful active principles for the preparation of pharmaceutical compositions intended to prevent or cure infections by fungi or parasites.

The compounds of the present invention differ from compounds of the prior art by the fact that they possess a central indole unit (or a related unit, such as indoline, azaindole, indazole), invariably substituted by a radical of the azolylalkyl type. The compounds of the invention are thus likely to be particularly advantageous in therapeutics in the treatment of mycoses and parasitoses.

Some of the compounds according to the invention are active against clinical strains of the fungus Aspergillus fumigatus which are resistant to conventional active principles such as itraconazole.

A first object of the invention consists of an antifungal and/or antiparasitic pharmaceutical composition comprising, as active principle, a compound of formula (I) below:

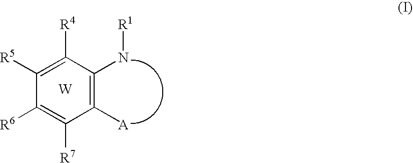

in which

A represents a bivalent radical chosen from among the following radicals:

(a) CR²═CR^{3,},

(b) CHR²—CHR³ or

(c) N═CR³, N being bound to the nitrogen atom of the NR¹ group represented in formula (I)

at least one of the radicals R¹ to R⁷ represents the linkage (CRR′)ₘ—(CR″R′″)ₙ—(CHX)ₚ-Het in which:

a)—R and R′ independently from each other represent hydrogen, a lower alkyl, alkenyl, cycloalkyl, phenyl, substituted phenyl, halogenophenylalkyl or benzotriazolyl group; or

R and R′ together form a saturated ring with five or six members, unsubstituted of substituted by a lower alkyl group or a halogen chosen from bromine, chlorine or fluorine;

b) R″ and R′″ independently from each other represent a lower alkyl, phenyl, substituted phenyl, halogenophenylalkyl, hydroxy, alkoxy or acyloxy group;

c) Het represents an 1H-imidazol-1-yl, 2-methyl-1H-imidazol-1-yl, 1H-1,2,4-triazol-1-yl, 4H-1,2,4-triazol-4-yl or 1H-tetrazol-5-yl group;

d) X represents a hydrogen or a lower alkyl, phenyl or substituted phenyl group;

e) m, n and p are independently from each other equal to 0, 1, 2, 3, 4 or 5;

the other radicals R¹ to R⁷ which are not part of the (CRR′)ₘ—(CR″R′″)ₙ—(CHX)ₚ-Het linkage independently from each other represent a hydrogen atom, a lower alkyl group, a halogen, a lower trifluoroalkyl, cyano, alkoxy, alkoxycarbonyl, carboxamido, phenyl, substituted phenyl, phenylalkyl or halogenophenylalkyl group.

the ring

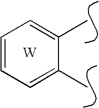

represents

either the phenyl nucleus, the central unit corresponding in this case to indole, indoline, indazole,

or the pyridine nucleus, the nitrogen being located in position 4, 5, 6 or 7 of the central bicyclic ring corresponding in this case to azaindole.

On condition that:

(1) when A represents N═CR³, the radical R¹ is different from a substituted benzyl radical or a substituted or unsubstituted ethyl radical and the radical R³ represents a hydrogen atom; and

(2) when A represents CR²═CR³, the radical R¹ is different from a substituted or unsubstituted benzyl radical and the radicals R² and R³ both represent a hydrogen atom.

(3) when A represents CR²═CR³ and the radical R³ represents Het-

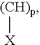

Het is different from a 1H-imidazol-1-yl ring,

or an enantiomer or a diastereoisomer of the compound of formula (I) or a salt from the addition to an acid of a compound of formula (I), in combination with a pharmaceutically acceptable vehicle.

By “alkyl”, “alkenyl” and “alkoxy” according to the invention, should be understood a straight or branched group of 1 to 9 carbon atoms.

By “lower alkyl”, “lower alkenyl” and “lower alkoxy” in the context of the invention, should be understood a straight or branched group of 1 to 6 carbon atoms.

By “cycloalkyl”” in the context of the invention, should be understood a five- or six membered saturated ring.

By “substituted phenyl” or “substituted phenylalkyl” according to the invention, should be understood a phenyl or phenylalkyl ring substituted on one or more carbon atoms of the ring by one or more groups chosen from among alkyl, alkoxy, trifluoromethyl, trifluoromethoxy, hydroxy, halogen, cyano, thiol and alkylthio.

Among the salts from the addition to an acid of a compound of formula (I) according to the invention, the preferred salts are from addition to an acid chosen from among hydrochloric, sulfuric, tartaric, maleic, fumaric, oxalic, methanesulfonic, camphoric, nitric and ethanesulfonic acids.

The invention also relates to an antifungal and/or antiparasitic pharmaceutical composition, characterized in that it comprises a compound of formula (IA) below:

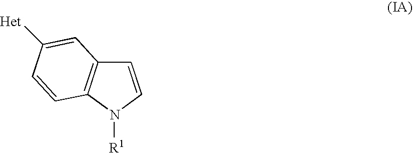

in which:

R¹ represents hydrogen or an alkyl, phenylalkyl, or substituted phenylalkyl group, and

Het represents an 1H-imidazol-1-yl, 1H-1,2,4-triazol-1-yl or 1H-tetrazol-5-yl group.

A first preferred compound of formula (IA) according to the invention is 1-(4-fluorobenzyl)-5-(1H-imidazol-1-yl)-1H-indole A second preferred compound of formula (IA) is 1-(4-fluorobenzyl)-5-(1H-tetrazol-5-yl)-1H-indole.

The invention also concerns an antifungal and/or antiparasitic pharmaceutical composition, characterized in that it comprises a compound of formula (IB) below:

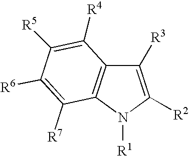

in which:

at least one of the radicals R² to R⁷ represents a group

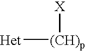

in which

X represents hydrogen, alkyl, phenyl, halogenophenyl and Het represents a 1H-imidazol-1-yl, 1H-1,2,4-triazol-1-yl or 4H-1,2,4-triazol-4-yl ring and p is equal to 0, 1, 2, 3, 4 or 5, with the exception of the compounds of formula (IB) in which R represents

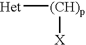

with Het representing a 1H-imidazol-1-yl ring,

the R² to R⁷ radicals not forming part of the group

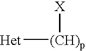

independently from each other represent a group chosen from among hydrogen, alkyl, alkoxy, alkoxycarbonyl, halogenoalkyl or cyano;

the radical R¹ represents hydrogen, or a phenyl, substituted phenyl, phenylalkyl or halogenophenylalkyl group.

The preferred compounds of formula (IB) are the following compounds:

1-(4-Fluorobenzyl)-2-(1H-imidazol-1-ylmethyl)-1H-indole;

5-Bromo-1-(4-chlorobenzyl)-3-(1H-imidazol-1-ylmethyl)-1H-indole;

1-(2-Chlorobenzyl)-3-(1H-1,2,4-triazol-1-ylmethyl)-1H-indole;

1-(2-Chlorobenzyl)-3-(4H-1,2,4-triazol-4-ylmethyl)-1H-indole;

Other preferred compounds of formula (IB) according to the invention are the following compounds:

1-Ethyl-2-[(4-fluorophenyl)(1H-imidazol-1-yl)methyl]-3-methyl-1H-indole;

5-Bromo-1-ethyl-2-[(4-fluorophenyl)(1H-1,2,4-triazol-1-yl)methyl]-3-methyl-1H-indole

5-Bromo-1-ethyl-3-[(4-fluorophenyl)(1H-imidazol-1yl)methyl]-1H-indole;

5-Bromo-1-ethyl-7-[(4-fluorophenyl)(1H-imidazol-1-yl)methyl]indoline;

5-[(4-Chlorophenyl)(1H-imidazol-1-yl)methyl]-1-ethyl-1H-indole;

The invention also relates to a pharmaceutical composition characterized in that the compound is of formula (IB1) below:

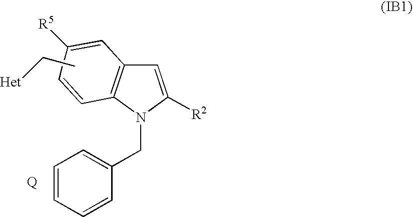

in which

R⁵ represents hydrogen, bromine, chlorine, fluorine or the methoxy group, R² represents hydrogen or the methyl group and Het represents the imidazolyl group bonded in the position 3, 4, 5 or 6 or the 1-triazolyl group bonded in position 3.

The invention further concerns a pharmaceutical composition characterized in that the compound is of formula (IB2) below:

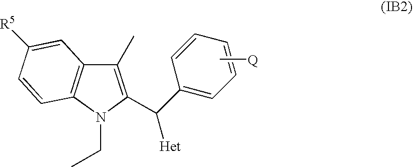

in which:

R⁵ represents hydrogen or bromine, Het represents an imidazolyl or 1-triazolyl group and Q represents one or two atoms of bromine or chlorine bonded to the positions 2, 3 or 4.

The invention also relates to a pharmaceutical composition characterized in that the compound is of formula (IB3) below:

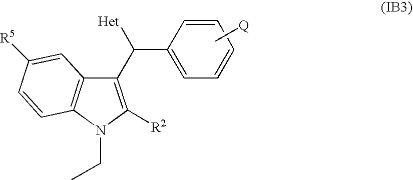

in which:

R² represents hydrogen or the methyl group, R⁵ represents hydrogen or a bromine atom, Het represents the imidazolyl group and Q represents one or two chlorine or fluorine atoms bonded to the positions 2, 3 or 4.

The invention also concerns a pharmaceutical composition characterized in that the compound is of formula (IB4) below:

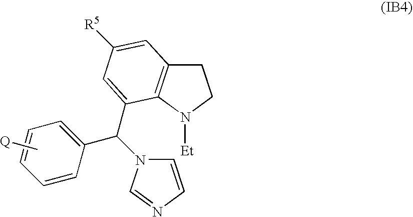

in which

R⁵ is hydrogen or a chlorine atom, Et represents the ethyl radical and Q represents a chlorine or fluorine atom bonded in position 3 or 4.

According to another embodiment, a pharmaceutical composition according to the invention is characterized in that the compound is of formula (IB5) below:

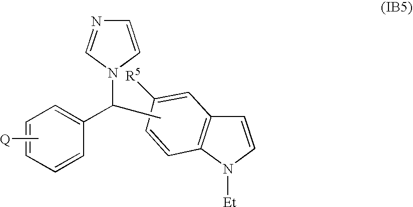

in which:

R⁵ represents hydrogen or bromine, Et represents the ethyl radical and Q represents a bromine, chlorine, or fluorine atom bonded in position 3 or 4.

The pharmaceutical composition of the invention may also be characterized in that it comprises a compound of formula (IC) below:

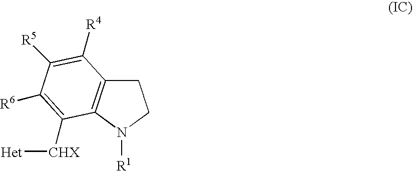

in which:

X represents hydrogen, alkyl, phenyl, halogenophenyl and Het represents an 1H-imidazol-1-yl, 1H-1,2,4-triazol-1-yl or 4H-1,2,4-triazol-4-yl ring;

the radicals R⁴ to R⁷ independently from each other represent a group chosen from among hydrogen, alkyl, alkoxy, alkoxycarbonyl, halogenoalkyl or cyano;

the radical R¹ represents hydrogen, or a phenyl, substituted phenyl, phenylalkyl or halogenophenylalkyl group.

According to a further embodiment, the invention concerns a pharmaceutical composition such as defined above, characterized in that it comprises a compound of formula (ID) below:

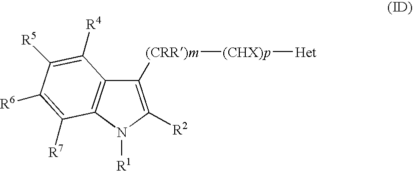

in which:

R³ represents the linkage (CRR′)ₘ—(CHX)ₚ-Het in which:

a)—R and R′ independently from each other represent hydrogen, a lower alkyl, alkenyl, cycloalkyl, phenyl, substituted phenyl, halogenophenylalkyl or benzotriazolyl group; or

R and R′ together form a five- or six-membered saturated ring, either unsubstituted or substituted by a lower alkyl group or a halogen chosen from among bromine, chlorine and fluorine.

c) Het represents an 1H-imidazol-1-yl, 2-methyl-1H-imidazol-1-yl, 1H-1,2,4-triazol-1-yl, 4H-1,2,4-triazol-4-yl or tetrazol-5-yl group;

d) X represents a hydrogen or a lower alkyl, phenyl or halogenophenyl group;

d) m and p are independently from each other equal to 0, 1, 2, 3, 4 or 5;

the radicals R¹, R², R⁴, R⁵, R⁶ and R⁷ represent, independently from each other, a hydrogen atom, a lower alkyl group, a halogen, a lower trifluoroalkyl, cyano, alkoxy, alkoxycarbonyl, carboxamido, phenyl, substituted phenyl, phenylalkyl or halogenophenylalkyl group.

Compounds of formula (ID) according to the invention are the following compounds:

1-(4-Chlorobenzyl)-3-(2-1H-imidazol-1-ylethyl)-1H-indole;

1-[1-(4-Chlorobenzyl)-1H-indol-3yl]-1-(1H-imidazol-1-ylmethyl)-cyclopentane;

1-(2,4-dichlorobenzyl)-3-[(1H-imidazol-1-yl)(methyl)methyl]-2-methyl-1H-indole.

The invention also covers a pharmaceutical composition as defined in the present description, characterized in that the compound is of formula (ID1) below:

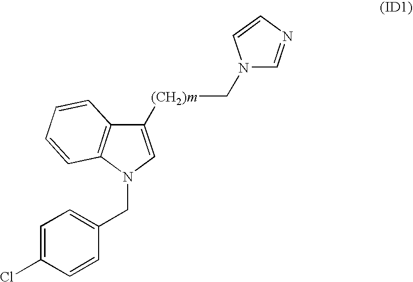

in which m is equal to 1, 2, 3, 4 or 5.

The invention also concerns a pharmaceutical composition such as defined above and which is characterized in that the compound is of formula (ID2) below:

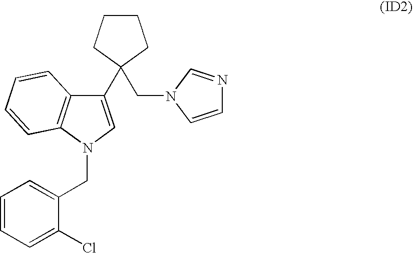

According to a further embodiment, the invention concerns a pharmaceutical composition characterized in that the compound is of formula (ID3) below:

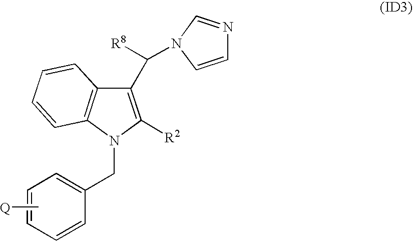

in which:

R⁸ represents a methyl, ethyl, or n-butyl group; and

Q represents one or two atoms of chlorine, bromine or fluorine and R² is as defined for the formula (ID).

Another object of the invention is a pharmaceutical composition such as defined above and characterized in that it comprises a compound of formula (IE) below:

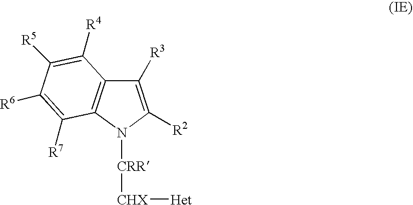

in which:

a)—R and R′ represent independently from each other hydrogen, a lower alkyl, alkenyl, cycloalkyl, phenyl, substituted phenyl, halogenophenylalkyl or benzotriazolyl group; or

R and R′ together form a five- or six-membered saturated ring, either unsubstituted or substituted by a lower alkyl group or a halogen chosen from among bromine, chlorine or fluorine;

b) Het represents a 1H-imidazol-1-yl, 2-methyl-1H-imidazol-1-yl, 1H-1,2,4-triazol-1-yl, 4H-1,2,4-triazol-4-yl or tetrazol-5-yl group;

c) X represents a hydrogen or a lower alkyl, phenyl or halogenophenyl group;

the radicals R² to R⁷ represent, independently from each other, a hydrogen atom, a lower alkyl group, a halogen, a lower trifluoroalkyl, cyano, alkoxy, alkoxycarbonyl, carboxamido, phenyl, substituted phenyl, phenylalkyl or halogenophenylalkyl group.

A compound corresponding to the formula (IE) above is the following compound:

1-[2-(4-Fluorophenyl)-2-(1H-imidazol-1-yl)-1-(benzotriazol-1-yl)ethyl]-1H-indole.

According to a further embodiment, the compound of formula (IE) is represented by the formula (IE1) below:

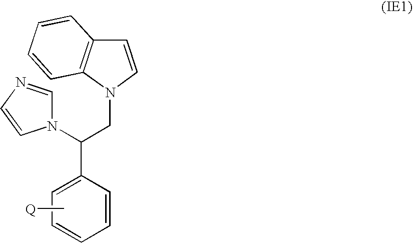

in which Q represents a chlorine or fluorine atom bonded to position 4.

The invention also relates to a pharmaceutical composition such as defined in the present description, characterized in that the compound is of formula (IF), below:

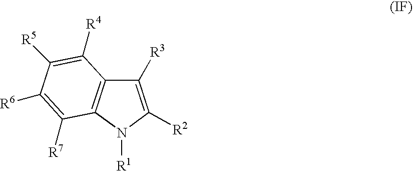

in which:

at least one of the radicals R¹ to R⁷ represents the linkage

Het-(CHX)—(CR″R′″)—(CRR′)ₘ

in which:

a) R and R′ represent independently from each other hydrogen or an alkyl group,

b) R″ represents a phenyl or substituted phenyl group,

c) R′″ represents hydrogen or a hydroxy, alkoxy or acyloxy group,

d) Het represents a 1H-imidazol-1-yl, 2-methyl-1H-imidazol-1-yl, 1H-1,2,4-triazol-1-yl, 4H-1,2,4-triazol-4-yl or tetrazol-5-yl group;

e) m is equal to 1 or 2;

f) X represents a hydrogen or a lower alkyl, phenyl or halogenophenyl group;

the other radicals R¹ to R⁷ which are not part of the linkage

Het-(CHX)—(CR″R′″)—(CRR′)ₘ

represent, independently from each other, a hydrogen atom, a lower alkyl, alkoxy, halogenoalkyl, or cyano group or a halogen atom.

According to another embodiment, the compound of formula (IF) is represented by the formula (IF1) below:

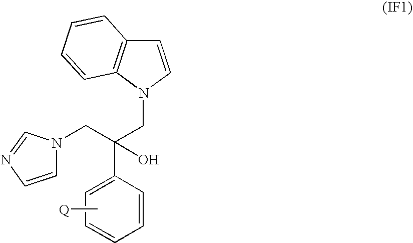

in which Q represents one or two chlorine or fluorine atoms bonded to positions 2 and/or 4.

A preferred compound corresponding to formula (IF) according to the invention is the following compound:

2-(4-Bromophenyl)-1-(1H-imidazol-1-yl)-3-(indol-1-yl)propan-2-ol;

The pharmaceutical composition according to the invention may also be characterized in that the compound is of formula (IG) below:

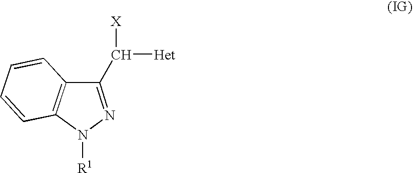

in which:

R¹ represents hydrogen, a substituted phenyl, phenylalkyl or substituted phenylalkyl group;

Het represents a 1H-imidazol-1-yl or 1H-1,2,4-triazol-1-yl radical;

X represents hydrogen, or an alkyl, phenyl or substituted phenyl group.

A preferred compound of formula (IG) according to the invention is 3-(1H-imidazol-1ylmethyl)-1-methyl-1H-indazole;

A further object of the invention is a pharmaceutical composition corresponding to the general technical properties above and characterized in that it comprises a compound of formula (IH) below:

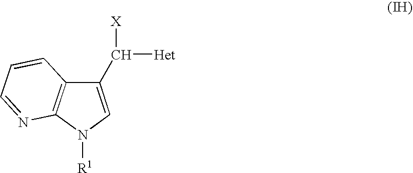

in which:

R¹ represents hydrogen, a substituted phenyl, phenylalkyl or substituted phenylalkyl group;

Het represents a 1H-imidazol-1-yl or 1H-1,2,4-triazol-1-yl radical;

X represents hydrogen, or an alkyl, phenyl or substituted phenyl group.

A preferred compound of formula (IH) is 1-(4-Fluorobenzyl)-3-(1H-imidazol-1ylmethyl)-1H-7-azaindole.

A pharmaceutical composition according to the invention is preferably in the form of a single dose comprising a quantity of the active principle of formula (I) necessary for the daily administration of 0.01 to 20 mg/kg of the active principle, preferably from 0.5 to 20 mg/kg.

As an example, a daily dose for a human of a pharmaceutical composition according to the invention may comprise from 0.8 mg to 1600 mg of an active principle of formula (I), preferably from 8 mg to 400 mg.

According to a first embodiment, a pharmaceutical composition according to the invention is characterized in that it is in a form suitable for oral administration.

According to a second embodiment, a pharmaceutical composition according to the invention is characterized in that it is in a form suitable for topical administration. According to a third embodiment, a pharmaceutical composition according to the invention is characterized in that it is in a form suitable for parenteral or intravenous administration.

A pharmaceutical composition according to the invention contains a pharmaceutically acceptable vehicle or pharmaceutically acceptable excipients, such as diluents or fillers. Such a pharmaceutical composition, preferably sterile, may be in the form of an aqueous or oily dispersion formulated with dispersing agents or wetting agents. A particular pharmaceutically acceptable vehicle and the ratio between the pharmaceutically acceptable vehicle and the compound of formula (I) according to the invention are determined with reference to the solubility and the chemical properties sought for in the composition, the method of administration and the regulatory practices in the pharmaceutical field.

The terms <<pharmaceutically acceptable >> or <<pharmaceutically compatible>> are used with reference to compounds and compositions which are physiologically tolerated, in other words which do not produce allergic reactions when administered to humans or animals.

The term <<excipient>>, in the context of the invention, describes a diluent, adjuvant, or vehicle with which the compound of formula (I) according to the invention is administered. Such pharmaceutical vehicles may be sterile liquids, such as water and oils, for example peanut, soya or sesame oil or a mineral oil. It is also possible to use water or aqueous saline solutions or aqueous solutions of dextrose and of glycerol, particularly for the preparation of injectable solutions.

A pharmaceutical composition according to the invention may be administered by an oral, rectal, parenteral, intravenous, subcutaneous or intradermal route.

Pharmaceutically acceptable excipients or vehicles are for example described in the book <<Remington's Pharmaceutical Sciences>> published by E. W. Martin, to which a person skilled in the art may advantageously refer.

The skilled person may refer to articles by JOLY et al. (1994) and by Georgopapadakov et al. (1996) or to the U.S. Pat. Nos. 5,545,652, 6,039,981, 5,846,971, 5,834,472 and 6,001,822 to produce an antifungal and/or antiparasitic composition according to the invention.

The invention also relates to a compound of formula (I) such as defined above, by way of a novel compound, as well as their enantiomers and diastereoisomers and their addition salts with acids.

The invention also covers novel compounds chosen from among the following novel compounds:

compounds of formula (IA) such as defined above;

compounds of formula (IB) such as defined above;

compounds of formula (IB1) such as defined above;

compounds of formula (IB2) such as defined above;

compounds of formula (IB3) such as defined above;

compounds of formula (IB4) such as defined above;

compounds of formula (IB5) such as defined above;

compounds of formula (IC) such as defined above;

compounds of formula (ID) such as defined above;

compounds of formula (ID1) such as defined above;

compounds of formula (ID2) such as defined above;

compounds of formula (ID3) such as defined above;

compounds of formula (IE) such as defined above;

compounds of formula (IE1) such as defined above;

compounds of formula (IF) such as defined above.

compounds of formula (IF1) such as defined above;

compounds of formula (IG) such as defined above; et

compounds of formula (IH) such as defined above.

A first family of preferred compounds according to the invention are the following compounds:

1-(4-fluorobenzyl)-5-(1H-imidazol-1-yl)-1H-indole; and

1-(4-fluorobenzyl)-5-(1H-tetrazol-5-yl)-1H-indole.

A second family of preferred compounds according to the invention are the following compounds:

1-(2-Chlorobenzyl)-3-(1H-1,2,4-triazol-1-ylmethyl)-1H-indole;

1-(2-Chlorobenzyl)-3-(4H-1,2,4-triazol-4-ylmethyl)-1H-indole;

A third family of preferred compounds according to the invention are the following compounds:

1-Ethyl-2-[(4-fluorophenyl)(1H-imidazol-1-yl)methyl]-3-methyl-1H-indole;

5-Bromo-1-ethyl-2-[(4-fluorophenyl)(1H-1,2,4-triazol-1-yl)methyl]-3-methyl-1H-indole;

5-Bromo-1-ethyl-3-[(4-fluorophenyl)(1H-imidazol-1yl)methyl]-1H-indole;

5-Bromo-1-ethyl-7-[(4-fluorophenyl)(1H-imidazol-1-yl)methyl]indoline; et

5-[(4-Chlorophenyl)(1H-imidazol-1-yl)methyl]-1-ethyl-1H-indole.

A fourth family of preferred compounds according to the invention are the following compounds

1-(4-Chlorobenzyl)-3-(2-1H-imidazol-1-ylethyl)-1H-indole;

1-[1-(4-Chlorobenzyl)-1H-indol-3yl]-1-(1H-imidazol-1-ylmethyl)-cyclopentane;

1-(2,4-dichlorobenzyl)-3-[(1H-imidazol-1-yl)(methyl)methyl]-2-methyl-1H-indole.

A fifth family of preferred compounds according to the invention are the following compounds:

2-(4-Bromophenyl)-1-(1H-imidazol-1-yl)-3-(indol-1-yl)propan-2-ol; and

1-[2-(4-Fluorophenyl)-2-(1H-imidazol-1-yl)-1-(benzotriazol-1-yl)ethyl]-1H-indole.

Other preferred compounds according to the invention are the following compounds:

3-(1H-Imidazol-1ylmethyl)-1-methyl-1H-indazole;

1-(4-Fluorobenzyl)-3-(1H-Imidazol-1ylmethyl)-1H-7-azaindole.

Another object of the invention is the use of a compound such as defined above for producing an antifungal and/or antiparasitic pharmaceutical composition.

According to a first embodiment, the use is characterized in that the pharmaceutical composition is in a form suitable for oral administration.

According to a second embodiment, the use is characterized in that the pharmaceutical composition is in a form suitable for topical administration.

According to a third embodiment, the use is characterized in that the pharmaceutical composition is in a form suitable for parenteral or intravenous administration.

A further object of the present invention is methods of preparation of the compounds of formula (I) such as defined in the present description, and particularly the compounds of formula (IA), (IB), (IC), (ID), (IE), (IF), (IG) and (IH). These methods of the invention are described below.

[symbol] Compounds Belonging to the Compounds of Formula (IA):

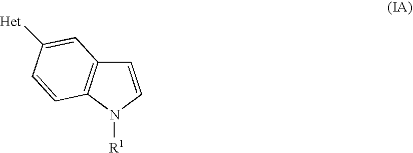

where R¹ corresponds to hydrogen, alkyl, substituted phenyl, phenylalkyl, substituted phenylalkyl,

where Het corresponds to 1H-imidazol-1-yl, 1,2,4-1H-triazol-1-yl or tetrazol-5-yl,

saccording to a method characterized in that an intermediate of formula (1) is used as the starting material:

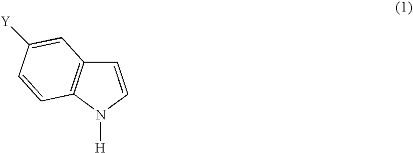

where Y represents halogen or a cyano group,

which is condensed with a derivative of formula (2):

R¹-Z (2)

where R¹ corresponds to alkyl, substituted phenyl, phenylalkyl or substituted phenylalkyl,

where Z corresponds to halogen or hydroxy,

to lead to a derivative of formula (3):

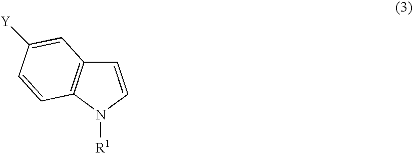

where Y and R¹ have the same definition as previously, and which is then condensed:

either with a sodium or potassium salt of the imidazole, when Y halogen,

or with sodium azide, when Y=cyano,

to give the compounds of formula (IA).

[symbol] Compounds of Formula (IB):

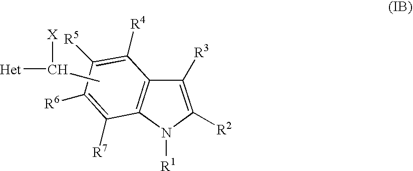

where R¹ corresponds to hydrogen, substituted phenyl, phenylalkyl or substituted phenylalkyl,

where R², R³, R⁴, R⁵, R⁶ and R⁷, in the absence of the linkage CHX-Het, correspond to hydrogen, alkyl, alkoxy, halogen, halogenoalkyl, halogenoalkoxy or cyano,

according to a method characterized in that the starting material used is:

either an indole derivative of formula (4):

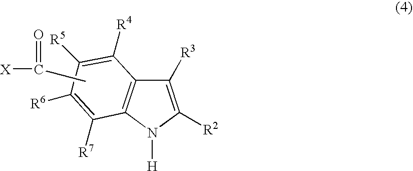

where X represents hydrogen or alkoxy,

which may be condensed with R¹Z (2), described above, to lead to a derivative of formula (5):

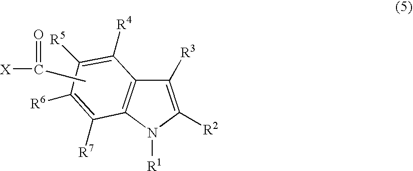

which [(4) and (5)] are reduced to alcohols immediately condensed with 1,1′-carbonyldiimidazole (CDI), 1,1′-carbonylditriazole (CDT) or 1,1′-sulfinylditriazole (SDT) to give the compounds of formula (IB),

or an acylindole of formula (6):

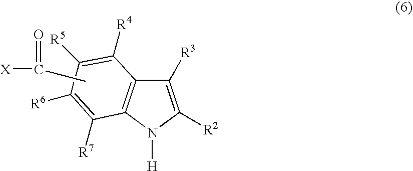

where X corresponds to alkyl, phenyl or substituted phenyl,

which may be condensed with R¹Z (2) described above to lead to a derivative of formula (7):

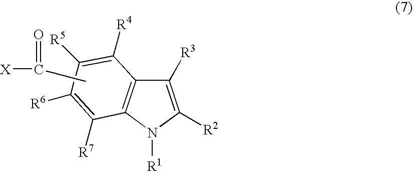

which [(6) and (7)] are then reduced to alcohols then condensed with CDI or SDT to give the compounds of formula (IB).

[symbol] Compounds of Formula (IC):

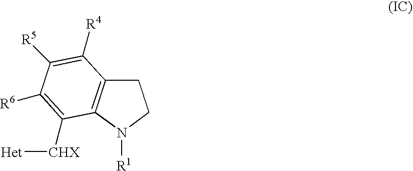

where R¹ is as described in (IB),

where R⁴, R⁵ and R⁶ correspond to hydrogen, alkyl, alkoxy, halogen, halogenoalkyl, halogenoalkoxy or cyano,

where X corresponds to hydrogen, alkyl, phenyl or substituted phenyl,

where Het corresponds to 1H-imidazol-1-yl or 1H-1,2,4-triazol-1-yl,

according to a method characterized in that an intermediate of formula (8) is used as starting material:

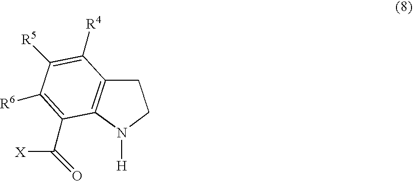

which may be condensed with R¹-Z (2) described above to lead to a derivative of formula (9):

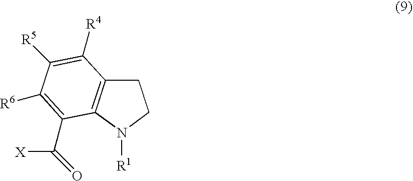

before being reduced to the alcohol then condensed with CDI or SDT to give the compounds of formula (IC).

[symbol] Compounds of Formula (ID):

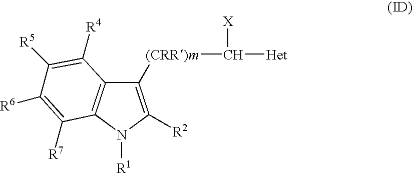

where R¹ corresponds to hydrogen, alkyl, phenyl, substituted phenyl, phenylalkyl or substituted phenylalkyl,

where R², R⁴, R⁵, R⁶ and R⁷ correspond to hydrogen, alkyl, phenyl, substituted phenyl, halogen, hydroxy, alkoxy, cyano, trifluoromethyl or trifluoromethoxy,

where CRR′ corresponds to cycloalkyl, phenylalkyl or substituted phenylalkyl,

where X and Het are as described for the compounds of formula (IC),

where m corresponds to 1, 2, 3,

according to a method using as starting material:

either the ester of formula (10):

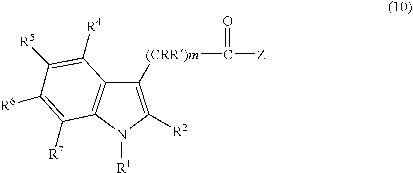

where Z corresponds to alkoxy,

or the ketone of formula (11):

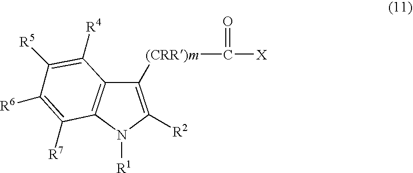

where X is as described in (IC),

which is reduced to the alcohol then condensed with CDI or SDT to give the compounds of formula (ID).

[symbol] Compounds of Formula (IE):

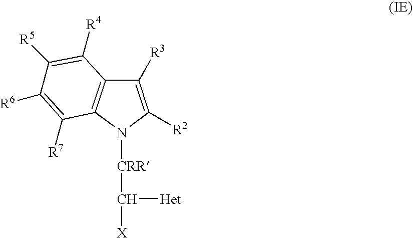

where R², R³, R⁴, R⁵, R⁶ and R⁷ have the same meaning as for the compounds of formula (ID),

where R, R′ represent hydrogen, alkyl or 1H-benzotriazol-1-yl,

where X represents hydrogen, alkyl, phenyl or halogenophenyl,

where Het is as described in (IC),

according to a method which uses as starting material an intermediate of formula (12):

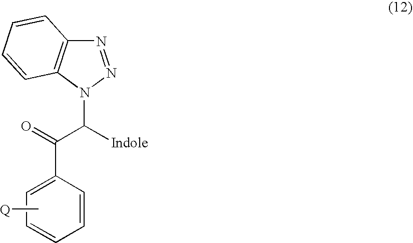

where Q corresponds to hydrogen, lower alkyl group, halogen, lower trifluoroalkyl, cyano, alkoxy or alkoxycarbonyl,

which is:

either, after elimination of the benzotriazole, leading to the intermediate of formula (13):

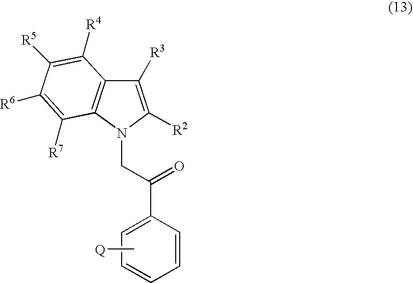

reduced to the alcohol then condensed with CDI or SDT,

or directly reduced to the alcohol then condensed with CDI or SDT,

to give the compounds of formula (IE).

[symbol] Compounds of Formula (IF):

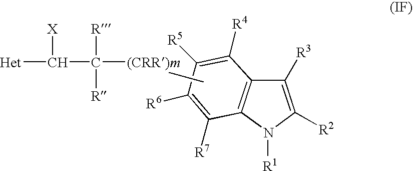

where R¹ to R⁷ may correspond to the linkage (CRR′)ₘ—CR″R′″-CHX-Het or are as described in the compounds of formula (IC):

R and R′ correspond to H or alkyl,

R″ corresponds to phenyl or substituted phenyl,

R′″ corresponds to hydrogen, hydroxy, alkoxy or acyloxy,

X corresponds to hydrogen or alkyl,

m corresponds to 1 or 2,

according to methods (a) or (b):

method (a) which uses as starting material a compound of formula (13) described above to obtain a compound of formula (14):

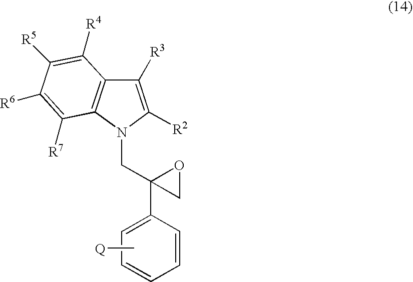

method (b) which uses as starting material a compound of formula (15)

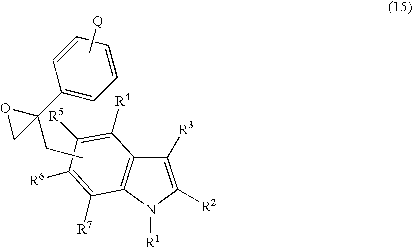

where Q corresponds to hydrogen, lower alkyl group, halogen, lower trifluoroalkyl, cyano, alkoxy, alkoxycarbonyl, which, after cleavage of the epoxide, gives the compounds of formula (IF).

[symbol] Compounds of Formula (IG):

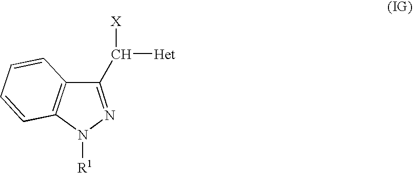

where R¹, X and Het are as described in (IC),

according to a method characterized in that an intermediate of formula (16) is used as starting material:

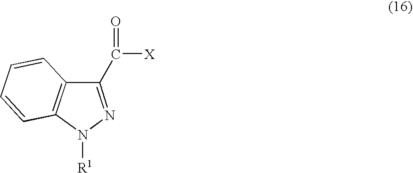

which is reduced to the alcohol then condensed with CDI or SDT to give the compounds of formula (IG).

[symbol] Compounds of Formula (IH):

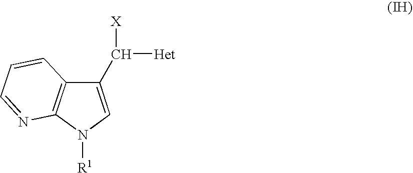

where R¹, X and Het are as described in (IC),

according to a method characterized in that an intermediate of formula (17) is used as starting material:

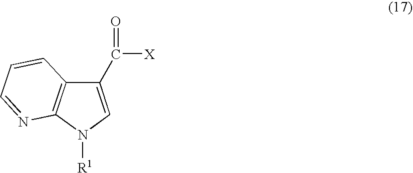

which is reduced to the alcohol than condensed with CDI or SDT to give the compounds of formula (IH).

Derivatives of formula (IA), (IB), (IC), (ID), (IE), (IF), (IG) and (IH) form the set of derivatives of formula (I), derivatives of formula (I) from which the enantiomers and diastereoisomers may be separated and which may be converted into salts by a pharmaceutically acceptable acid.

The present invention is further illustrated, without in any way being limited, by the figures and the following examples.

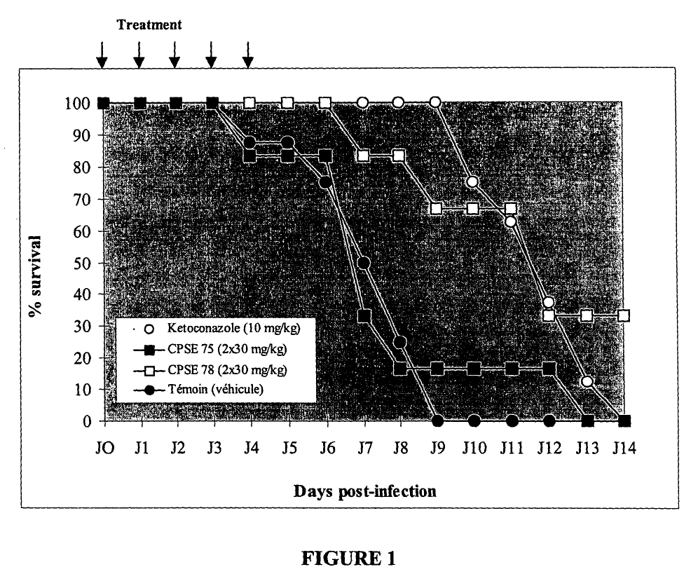

FIG. 1 illustrates the results of an in vivo test of the antifungal activity of compounds n°75 and n°78 of the invention. The abscissa shows the time after infection of the mice in days. The ordinate represents the percentage survival of the mice.

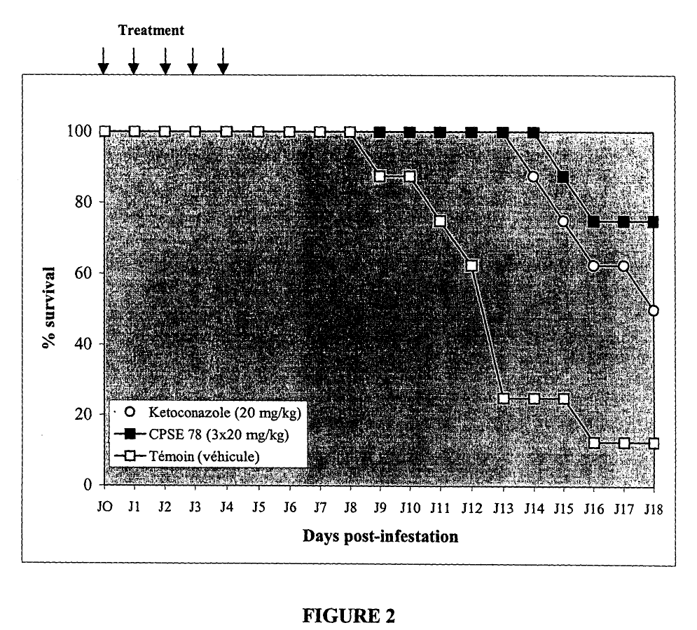

FIG. 2 illustrates the results of an in vivo test of the antifungal activity of compound n°78.

### EXAMPLES

General Methodology

The ¹H nuclear magnetic resonance spectra were performed using TMS (tetramethylsilane) as internal standard. The chemical shifts are expressed in parts per million (p.p.m.). The infrared spectra were performed either in the form of potassium bromide discs containing about 1% du product to be analysed or in the form of a film deposited on a sodium chloride plate.

The starting materials used were either commercially available or could be obtained by a person skilled in the art making use of the literature and preparations which do not form part of the invention but which are useful for preparing certain products of the invention.

The preparations form part of the invention but are useful for performing the synthesis of the derivatives of the invention.

Synthesis of the Compounds of Formula (IA)

1. Preparation of the Intermediates

### Example 1

### 5-Bromo-1-(4-fluorobenzyl)-1H-indole

In 30 ml of anhydrous dimethylformamide, place 2.4 g (60 mmol) of sodium hydride in 60% suspension in mineral oil. Stir and heat the mixture to a temperature of 30° C. Add progressively, using a spatula, 4.0 g (20 mmol) of 5-bromo-1H-indole. Continue the heating for 1 hour (until no further hydrogen is evolved). Cool the mixture to ambient temperature and add 3.18 g (22 mmol) of 4-fluorobenzyl chloride. Heat the mixture again to 30° C. for 30 minutes. Add water, extract with dichloromethane, dry the organic phase over anhydrous sodium sulfate and evaporate. Purify the evaporation residue by chromatography on silica gel with elution by dichloromethane (Int. 1).

Yield: 97%

Yellow oil

### Example 2

### 5-Cyano-1-(4-fluorobenzyl)-1H-indole

In 15 ml of anhydrous dimethylformamide, place 0.14 g (5.82 mmol) of sodium hydride in 60% suspension in mineral oil. Stir and heat the mixture to a temperature of 30° C. Add progressively, using a spatula, 0.33 g (1.94 mmol) of 5-cyano-1H-indole. Continue the heating for 1 hour (until no further hydrogen is evolved). Cool the mixture to ambient temperature and add 0.42 g (2.91 mmol) of 4-fluorobenzyl chloride. Heat the mixture again to 30° C. for 30 minutes. Add water, extract with dichloromethane, dry the organic phase over anhydrous sodium sulfate and evaporate. Purify the evaporation residue by chromatography on silica gel with elution by dichloromethane (Int. 2).

Yield: 95%

Orange oil

2. Preparation of the Final Compounds

### Example 3

### 1-(4-Fluorobenzyl)-5-(1H-imidazol-1-yl)-1H-indole

In 20 mL of anhydrous dimethylformamide, place 0.79 g (19.74 mmol) of sodium hydride in 60% suspension in mineral oil. Stir at ambient temperature and add progressively, using a spatula, 1.34 g (19.74 mmol) of 1H-imidazole. Stir for 1 hour (until no further hydrogen is evolved). Add 86 mg of copper and 2 g (6.58 mmol) of 5-bromo-1-(4-fluorobenzyl)-1H-indole then heat the reaction mixture to 150° C. for 48 hours. Hydrolyse the reaction medium, extract with dichloromethane, dry the organic phase over anhydrous sodium sulfate and evaporate. Purify the evaporation residue by chromatography on silica gel with elution by a 19/1 dichloromethane/absolute ethanol mixture (Cpsé 1).

Yield: 11%

Melting point: 121-122° C. (dichloromethane/absolute ethanol)

### Example 4

### 1-(4-Fluorobenzyl)-5-(1H-tetrazol-5-yl)-1H-indole

Successively introduce 0.63 g (2.52 mmol) of 5-cyano-1-(4-fluorobenzyl)-1H-indole, 0.22 g (4.15 mmol) of ammonium chloride and 0.35 g (4.15 mmol) of sodium azide in 30 mL of anhydrous tetrahydrofuran into a flask. Heat the reaction medium to 120° C. for 18 hours. Evaporate the solvent and add 35 mL of water and 7 mL of concentrated hydrochloric acid. Extract with ethyl acetate and wash the organic phase with a saturated sodium chloride solution. Evaporate the solvent (Cpsé 2).

Yield: 45%

Melting point: 183-186° C. (ethyl acetate)

Synthesis of Compounds of Formula (IB) and (IC)

I. Sub-Series With Straight Chain

I.1 Preparation of the Intermediates

### Example 5

### 2-Ethoxycarbonyl-1-(4-fluorobenzyl)-1H-indole

1a) 2-Ethoxycarbonyl-1H-indole

In 50 mL of a solution of ethanol containing 5% hydrochloric acid, place 2 g (12.4 mmol) of indole-2-carboxylique acid. Stir and bring to reflux for 24 hours. Concentrate, cool and isolate the ester by filtration (Int. 3).

Yield: quantitative

Melting point: 110-114° C. (absolute ethanol)

1b) 2-Ethoxycarbonyl-1-(4-fluorobenzyl)-1H-indole

In 20 mL of acetonitrile, place 1.07 g (5.6 mmol) of ethyl indole-2-carboxylate and 3.68 g (12.3 mmol) of anhydrous cesium carbonate. Stir and bring to reflux for 2 hours. Then add 0.98 g (6.8 mmol) of 4-fluorobenzyl chloride. Continue the heating for 1.5 hour. Extract with dichloromethane, dry over anhydrous sodium sulfate and evaporate to dryness. Take up the residue in diisopropyl ether, triturate and filter. (Int. 4).

Yield: 93%

Melting point: 78-79° C. (diisopropyl ether)

### Example 6

### 1-(2-Fluorobenzyl)-3-formyl-1H-indole

1a) 3-Formyl-1H-indole

Into a three-necked flask fitted with a stirrer and an immersion thermometer, and placed in an ice-salt bath, introduce 8.5 mL (110 mmol) of dry dimethylformamide. Cool the dimethylformamide and add over 30 minutes 2.61 mL (28 mmol) of phosphoryl chloride. Then add, over 40 minutes, the solution of 3 g (25.5 mmol) of 1H-indole in 5 mL of anhydrous dimethylformamide, making sure that the temperature does not rise above 10° C. Stir the mixture for 45 minutes at 10° C. then for 40 minutes at 35° C. Add 10 g of crushed ice, stir the compact mixture vigorously and add a further 10 g of crushed ice. Continue the stirring and add progressively, by a dropping funnel, a solution of 11.3 g (282 mmol) of sodium hydroxide in 30 mL of water, slowly at first, then more rapidly, maintaining a good level of stirring. Then bring the solution to the boil for 15 minutes, recover by filtration and wash the isolated 3-formyl-1H-indole several times with water. (Int. 5).

Yield: 98%

Melting point: 174-175° C. (absolute ethanol)

1b) 1-(2-Fluorobenzyl)-3-formyl-1H-indole

In 20 mL of acetonitrile, introduce 1.5 g (10 mmol) of 1H-indole-3-carbaldehyde and 6.52 g (20 mmol) of cesium carbonate. Stir and bring to reflux for 2 hours. Add 1.59 g (11 mmol) of 2-fluorobenzyl chloride. Maintain the reflux for 1 hour. Filter the solution and evaporate the solvent. Take up the residue in water, extract with dichloromethane, dry the organic phase over anhydrous sodium sulfate and evaporate (Int. 6).

Yield: 88%

Melting point: 95-97° C. (diethyl ether)

TABLE 1
Intermediates according to example 6

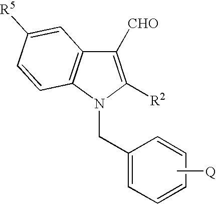

| N° Int. | Q | R² | R⁵ | Yld (%) | Mp (° C.) |
|---|---|---|---|---|---|
| 7 | 3-F | H | H | 87 | 105-107 (diisopropyl ether) |
| 8 | 2,4-diF | H | H | 83 | 120-121 (diisopropyl ether) |
| 9 | 4-tBu | H | H | 79 | 119-120 (diisopropyl ether) |
| 10 | 4-F | CH₃ | H | 75 | 121-123 (diisopropyl ether/ |
| | | | | | dichloromethane) |
| 11 | 2-Cl | H | Br | 80 | 128-129 (diisopropyl ether) |
| 12 | 4-Cl | H | Br | 55 | 200-201 (diisopropyl ether |
| 13 | 2-F | H | Br | 70 | 131-132 (diisopropyl ether) |
| 14 | 4-F | H | Br | 76 | 132-133 (diisopropyl ether) |
| 15 | 4-CN | H | Br | 98 | 159-160 (diisopropyl ether/ |
| | | | | | dichloromethane) |
| 16 | 4-Cl | H | Cl | 96 | 132-133 (diisopropyl ether) |
| 17 | 4-Cl | H | F | 81 | 151-152 (diisopropyl ether) |
| 18 | 4-F | H | OCH₃ | 46 | 114-115 (diisopropyl ether) |

### Example 7

### 1-(4-Fluorobenzyl)-4-methoxycarbonyl-1H-indole

1a) Methyl 2-(2-dimethylaminoethenyl)-3-nitrobenzoate

Under a nitrogen atmosphere, to a suspension of 10 g (51.23 mmol) of methyl 2-methyl-3-nitrobenzoate in 30 ml of dimethylformamide, add 11.36 mL (85.55 mmol) of dimethylformamide dimethylacetal. Heat to 110° C. for 6 hours. Add one litre of water. Extract with diethyl ether, wash the organic phase several times with water. Dry over anhydrous sodium sulfate, filter and evaporate the solvent (Int. 19).

Yield: quantitative

Red oil

1b) 4-Methoxycarbonyl-1H-indole

Place under 5 bars of hydrogen a solution of 12.82 g (51.23 mmol) of methyl 2-(2-dimethylaminoethenyl)-3-nitrobenzoate in 80 mL of benzene. Add 8.53 g of 5% palladium on charcoal. Maintain the stirring, at ambient temperature, for 3 hours. Filter over celite. Evaporate the filtrate. Purify the evaporation residue by chromatography on silica gel with elution by a 9/1 hexane/ethyl acetate mixture. Evaporate the solvent (Int. 20).

Yield: 60%

Melting point: 66-67° C. (hexane)

1c) 1-(4-Fluorobenzyl)-4-methoxycarbonyl-1H-indole

In 30 mL of anhydrous dimethylformamide, place 0.96 g (40 mmol) of sodium hydride in 60% suspension in mineral oil. Stir and heat the mixture to a temperature of 30° C. Add progressively, using a spatula, 3.5 g (20 mmol) of 4-methoxycarbonyl-1H-indole. Continue the heating for 1 hour (until no further hydrogen is evolved). Cool the mixture to ambient temperature and add 3.18 g (22 mmol) of 4-fluorobenzyl chloride. Heat the mixture again to 30° C. for 30 minutes. Add water, extract with dichloromethane, dry the organic phase over anhydrous sodium sulfate and evaporate. Purify the evaporation residue by chromatography on silica gel with elution by dichloromethane (Int. 21).

Yield: 96%

Yellow oil

TABLE 2
Intermediates according to example 7

| N° | | | |
|---|---|---|---|
| Int. | Structure | Yld (%) | Mp (° C.) |
| 24 | 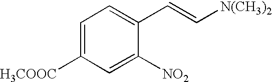 | quanti- tative | 121-123 (ethanol) |
| 25 | 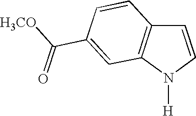 | 48 | 80-82 (ethyl acetate) |
| 26 | 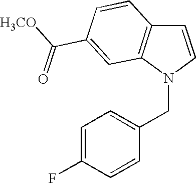 | 90 | 80-81 (hexane) |

### Example 8

### 1-(4-Fluorobenzyl)-5-formyl-1H-indole

1a) Methyl 1H-indole-5-carboxylate

Heat to 130° C. (oil bath) a solution of 10 g (55 mmol) of 3-methyl-4-nitrobenzoic acid and 20.26 g (170 mmol) of dimethylformamide of dimethylacetal in 50 mL of anhydrous dimethylformamide (the solution becomes red). Cool and add ethyl acetate. Wash the organic phase with a saturated aqueous sodium chloride solution. Dry over anhydrous sodium sulfate. Filter and evaporate the solvent to dryness. Isolate a red-brown powder. Place under 50 psi (3.5 bars) of hydrogen, for 26 hours, a solution of 12.38 g of methyl 3-(2-dimethylaminoethenyl)-4-nitrobenzoate in 200 mL of anhydrous benzene containing 2.45 g of 10% palladium on charcoal. Stir the solution. The solution changes from bright red to a very pale red. Filter over celite. Evaporate the solvent. Purify the evaporation residue by chromatography over silica gel with elution by dichloromethane (Int. 22).

Yield: 58%

Melting point: 122-123° C. (dichloromethane)

1b) Methyl 1-(4-fluorobenzyl)-1H-indole-5-carboxylate

In 10 mL of anhydrous dimethylformamide, place 0.31 g (7.8 mmol) of sodium hydride in 60% suspension in mineral oil. Stir and heat to a temperature of 30° C. Add progressively, using a spatula, 0.45 g (2.6 mmol) of methyl 1H-indole-5-carboxylate. Continue stirring and heating for 1 hour (until no further hydrogen is evolved). Cool the mixture to ambient temperature and add 0.42 g (2.9 mmol) of 4-fluorobenzyl chloride. Heat the mixture again to 30° C. for 30 minutes. Hydrolyse the reaction medium. Extract with dichloromethane, dry the organic phase over anhydrous sodium sulfate and evaporate (Int. 23).

Yield: 92%

Yellow oil

I.2. Preparation of th Final Compounds

### Example 9

### 1-(4-Fluorobenzyl)-2-(1H-imidazol-1-ylmethyl)-1H-indole

In 10 mL of anhydrous tetrahydrofuran, place 0.39 g (10.4 mmol) of lithium aluminium hydride. In a dropping funnel, place 1.55 g (5.2 mmol) of ethyl 1-(4-fluorobenzyl)-1H indole-2-carboxylate in 10 mL of tetrahydrofuran and add progressively to the mixture. Stir 20 minutes at ambient temperature. Add 10 ml of ethyl acetate, then 10 mL of water with a Pasteur pipette. Evaporate and extract with diethyl ether. Dry over anhydrous sodium sulfate, filter and evaporate to dryness.

In 15 ml of anhydrous tetrahydrofuran, place 1.29 g (5.1 mmol) of 1-(4-fluorobenzyl)-2-hydroxymethyl-1H-indole and 0.82 g (5.1 mmol) of 1,1′-carbonyldiimidazole. Stir and bring to reflux overnight. Extract the reaction medium with dichloromethane, dry the organic phase over anhydrous sodium sulfate and concentrate. Purify the evaporation residue by chromatography on silica gel with elution by a 19/1 dichloromethane/absolute ethanol mixture. The nitrate is obtained after addition of a solution of 0.06 g (0.6 mmol) of 68% nitric acid (in 20 mL of diethyl ether) to an ether solution (20 mL) containing 0.2 g (0.6 mmol) of the imidazole derivative. Filter the nitrate (Cpsé 3n).

Yield: 20%

Melting point: 155-156° C. (diethyl ether)

TABLE 3

| | |
|---|---|
| | (IB1) |
| 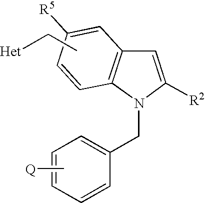 | |

| N° | | | | | Site of | Derv | Yld | | According |
|---|---|---|---|---|---|---|---|---|---|
| Cpsé | Q | R² | R⁵ | Het | fixation | from | (%) | Mp (° C.) | to ex. |
| 4 | 2-F | H | H | Imid | 3 | 6 | 42 | 65-68 | 10 |
| | | | | | | | | (diisopropyl ether) | |
| 5 | 3-F | H | H | Imid | 3 | 7 | 92 | 62-65 | 10 |
| | | | | | | | | (cyclohexane) | |
| 6 | 2,4-diF | H | H | Imid | 3 | 8 | 30 | 62-63 | 10 |
| | | | | | | | | (diisopropyl ether) | |
| 7 | 4-tBu | H | H | Imid | 3 | 9 | 48 | oil | 10 |
| 8 | 4-F | CH₃ | H | Imid | 3 | 10 | 30 | 157-158 | 10 |
| | | | | | | | | (diisopropyl ether) | |
| 9 | 2-Cl | H | Br | Imid | 3 | 11 | 50 | 98-100 | 10 |
| | | | | | | | | (diisopropyl ether) | |
| 11 | 2-F | H | Br | Imid | 3 | 13 | 26 | 101-102 | 10 |
| | | | | | | | | (diisopropyl ether) | |
| 12 | 4-F | H | Br | Imid | 3 | 14 | 34 | 123-124 | 10 |
| | | | | | | | | (diisopropyl ether) | |
| 13 | 4-CN | H | Br | Imid | 3 | 15 | 26 | 159-161 | 10 |
| | | | | | | | | (diisopropyl | |
| | | | | | | | | ether) | |
| 14 | 4-Cl | H | Cl | Imid | 3 | 16 | 26 | 135-136 | 10 |
| | | | | | | | | (diisopropyl | |
| | | | | | | | | ether) | |
| 15 | 4-Cl | H | F | Imid | 3 | 17 | 36 | 133-134 | 10 |
| | | | | | | | | (diisopropyl | |
| | | | | | | | | ether) | |
| 16 | 4-F | H | OCH₃ | Imid | 3 | 18 | 22 | 96-98 | 10 |
| | | | | | | | | (diisopropyl | |
| | | | | | | | | ether) | |
| 17 | 4-F | H | H | Imid | 4 | 21 | 51 | 94-96 | 9 |
| | | | | | | | | (diisopropyl | |
| | | | | | | | | ether) | |
| 18 | 4-F | H | H | Imid | 5 | 23 | 20 | 50 | 9 |
| | | | | | | | | (diisopropyl | |
| | | | | | | | | ether) | |
| 19 | 4-F | H | H | Imid | 6 | 26 | 51 | oil | 9 |
| 22 | 2-Cl | H | Br | 1-Triazol | 3 | 11 | 16 | 121-122 | 11 |
| | | | | | | | | (ethanol) | |
| 23 | 4-Cl | H | Br | 1-Triazol | 3 | 12 | 10 | 132-133 | 11 |
| | | | | | | | | (ethanol) | |
| 24 | 2-F | H | Br | 1-Triazol | 3 | 13 | 20 | 119-121 | 11 |
| | | | | | | | | (ethanol) | |
| 25 | 4-F | H | Br | 1-Triazol | 3 | 14 | 25 | 138-139 | 11 |
| | | | | | | | | (ethanol) | |

### Example 10

### 5-Bromo-1-(4-chlorobenzyl)-3-(1H-imidazol-1-ylmethyl)-1H-indole

In 20 mL of methanol, introduce 2.58 g (7.4 mmol) of 5-bromo-1-(4-chlorobenzyl)-3-formyl-1H-indole. Stir at ambient temperature. Add progressively, using a dropping funnel, a solution of 0.9 g (23.8 mmol) of sodium borohydride in 15 mL of methanol. Stir at ambient temperature for 1 hour. Hydrolyse the reaction medium, extract with dichloromethane, dry the organic phase over anhydrous sodium sulfate and evaporate to dryness.

In 20 ml of anhydrous tetrahydrofuran, introduce 1.30 g (3.7 mmol) of 5-bromo-1-(4-chlorobenzyl)-3-hydroxymethyl-1H-indole and 0.9 g (5.52 mmol) of 1,1′-carbonyldiimidazole. Stir and bring to reflux for 4 hours. Concentrate the solution and take up the residue in water. Extract with dichloromethane, dry the organic phase over anhydrous sodium sulfate and evaporate. Purify the evaporation residue by chromatography on silica gel with elution by a 19/1 dichloromethane/absolute ethanol mixture: (Cpsé 10).

Yield: 42%

Melting point: 45-47° C. (diisopropyl ether)

### Example 11

### 1-(2-Chlorobenzyl)-3-(1H-1,2,4-triazol-1-ylmethyl)-1H-indole and 1-(2-Chlorobenzyl)-3-(4H-1,2,4-triazol-4-ylmethyl)-1H-indole

In 20 mL of methanol, introduce 2 g (7.4 mmol) of 1-(2-chlorobenzyl)-3-formyl-1H-indole. Stir at ambient temperature. Add progressively, using a dropping funnel, a solution of 0.9 g (23.8 mmol) of sodium borohydride in 15 mL of methanol. Stir at ambient temperature for one hour. Hydrolyse the reaction medium, extract with dichloromethane, dry the organic phase over anhydrous sodium sulfate and evaporate to dryness.

In 20 mL of anhydrous tetrahydrofuran, introduce 1.0 g (3.7 mmol) of 1-(2-chlorobenzyl)-3-hydroxymethyl-1H-indole and 0.60 g (3.7 mmol) of carbonylditriazole. Stir and bring to reflux for 15 hours. Concentrate the solution and take up the residue in water. Extract with dichloromethane, dry the organic phase over anhydrous sodium sulfate and evaporate. Purify the evaporation residue by chromatography on silica gel with elution by a 19/1 dichloromethane/absolute ethanol mixture: (Cpsés 20 and 21).

Cpsé 20:

Yield: 74%

Melting point: 65-66° C. (cyclohexane/dichloromethane)

Cpsé 21:

Yield: 6%

Melting point: 130° C. (diisopropyl ether)

II. Sub-Series With Branched Chain

II.1. Preparation of the Intermediates

### Example 12

### 1-Ethyl-2-(4-fluorobenzoyl)-3-methyl-1H-indole

1a) 1-Ethyl-3-methyl-1H-indole

In 30 mL of anhydrous dimethylformamide, place 0.61 g (15 mmol) of sodium hydride in 60% suspension in mineral oil. Stir and heat the mixture to a temperature of 40° C. Add progressively, using a spatula, 1.0 g (7.6 mmol) of 3-methyl-1H-indole. Continue the heating for 30 minutes (until no further hydrogen is evolved). Cool the mixture to ambient temperature and add 1.42 g (9.1 mmol) of iodoethane. Heat the mixture again to 40° C. for 30 minutes. Add water, extract with dichloromethane, dry the organic phase over anhydrous sodium sulfate and evaporate. Purify the evaporation residue by chromatography on silica gel with elution by dichloromethane (Int. 27).

Yield: 95%

Yellow liquid

TABLE 4
Intermediate according to example 12-1a

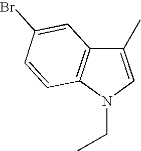

| N° Int. | Yld (%) | Mp (° C.) |
|---|---|---|
| 28 | 92 | — |

1b) 1-Ethyl-2-(4-fluorobenzoyl)-3-methyl-1H-indole

In 10 mL of dichloromethane, place 1.17 g (8.8 mmol) aluminium chloride and cool the solution by an ice bath. Add progressively, using a dropping funnel, 1.40 g (8.8 mmol) of 4-fluorobenzoyl chloride. Stir for one hour at 25° C. then add progressively, using a dropping funnel, a solution of 1.16 g (7.3 mmol) of 1-ethyl-3-methyl-1H-indole in 10 mL of dichloromethane. Bring the mixture to reflux for 6 hours. Pour the reaction medium into a solution of iced water and ethyl acetate. Extract with ethyl acetate, dry the organic phase over anhydrous sodium sulfate and evaporate. Purify the evaporation residue by chromatography on silica gel with elution by dichloromethane (Int. 34).

Yield: 70%

Yellow oil

TABLE 5
Intermediates according to Example 12

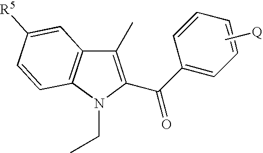

| N° Int. | Q | R⁵ | Derv from | Yld (%) | Mp (° C.) |
|---|---|---|---|---|---|
| 29 | 3-Br | H | 27 | 55 | oil |
| 30 | 4-Br | H | 27 | 45 | oil |
| 31 | 3-Cl | H | 27 | 36 | oil |
| 32 | 4-Cl | H | 27 | 36 | oil |
| 33 | 2,4-diCl | H | 27 | 31 | oil |
| 35 | 3-Cl | Br | 28 | 80 | oil |
| 36 | 4-Cl | Br | 28 | 76 | oil |
| 37 | 2,4-diCl | Br | 28 | 72 | 110-112 |
| | | | | | (diisopropyl |
| | | | | | ether) |
| 38 | 4-F | Br | 28 | 55 | 120-121 |
| | | | | | (diisopropyl |
| | | | | | ether) |

### Example 13

### 3-(4-Chlorobenzoyl)-1-ethyl-1H-indole

1a) 3-(4-Chlorobenzoyl)-1H-indole

In 20 mL of dichloromethane, place 2.29 g (17.22 mmol) of aluminium chloride and stir at ambient temperature. Add progressively, using a dropping funnel, a solution of 3.01 g (17.22 mmol) of 4-chlorobenzoyl chloride in 20 mL of dichloromethane. Stir for 1 hour at ambient temperature. Add progressively, using a dropping funnel, a solution of 2 g (17.22 mmol) of 1H-indole in 10 mL of dichloromethane. Stir for 48 hours at 25° C. Filter the reaction mixture. Pour the filtrate into a mixture of iced water and ethyl acetate. Extract with ethyl acetate, dry the organic phases over anhydrous sodium sulfate and evaporate (Int. 41).

Yield: 30%

Melting point: 230° C. (diisopropyl ether)

1b) 3-(4-Chlorobenzoyl)-1-ethyl-1H-indole

In 20 mL acetonitrile, introduce 0.6 g (2.34 mmol) of 3-(4-chlorobenzoyl)-1H-indole and 1.5 g (4.69 mmol) of cesium carbonate. Stir and bring to reflux for 2 hours. Add 0.43 g (2.8 mmol) of iodoethane. Maintain the reflux for 1 hour. Filter the solution and evaporate the solvent. Take up the residue in water, extract with dichloromethane, dry the organic phase over anhydrous sodium sulfate and evaporate (Int. 42).

Yield: 75%

Melting point: 99-100° C. (diisopropyl ether)

### Example 14

### 5-Bromo-1-ethyl-3-(4-fluorobenzoyl)-1H-indole

1a) 5-Bromo-3-(4-fluorobenzoyl)-1H-indole

Under a nitrogen atmosphere, introduce 0.13 g (5.6 mmol) of magnesium into a flask and cover the metal with anhydrous diethyl ether. Add progressively to the reaction medium, using a dropping funnel, 0.18 g (5.6 mmol) of iodoethane. Prepare a solution of 1.0 g (5.1 mmol) of 5-bromo-1H-indole and of 1.39 g (10.2 mmol) of zinc chloride in 15 ml of dichloromethane and add it slowly to the Grignard reagent. Stir the solution for one hour at ambient temperature then add 1.02 g (6.4 mmol) of 4-fluorobenzoyl chloride. Stir the solution for 1 hour at ambient temperature and add 0.13 g (2.3 mmol) of aluminium chloride. Stir the solution for 6 hours at ambient temperature. Pour the reaction medium over 25 mL of a saturated solution of ammonium chloride. Wash the organic phase with a saturated solution of sodium bicarbonate, dry it over anhydrous sodium sulfate, filter and evaporate to dryness. (Int. 51).

Yield: 43%

Melting point: 278° C. (diisopropyl ether)

1b) 5-Bromo-1-ethyl-3-(4-fluorobenzoyl)-1H-indole.

In 20 mL of acetonitrile, introduce 1 g (3.15 mmol) of 5-bromo-3-(4-fluorobenzoyl)-1H-indole and 2.05 g (6.3 mmol) of cesium carbonate. Stir and bring to reflux for 2 hours. Add 0.6 g (3.85 mmol) of iodoethane. Maintain the reflux for 1 hour. Filter the solution and evaporate the solvent. Take up the residue in water, extract with dichloromethane, dry the organic phase over anhydrous sodium sulfate and evaporate (Int. 52).

Yield: 88%

Melting point: 155-157° C. (diisopropyl ether/methanol)

### Example 15

### 5-Bromo-3-(2,4-dichlorobenzoyl)-1-ethyl-1H-indole

1a) 5-Bromo-1-ethyl-1H-indole

In 75 mL of anhydrous dimethylformamide, place 3.06 g (76.5 mmol) of sodium hydride in 60% suspension in mineral oil. Stir and heat the mixture to 80° C. Add progressively, using a spatula, 5.0 g (25.5 mmol) of 5-bromo-1H-indole. Continue the heating for 30 minutes (until no further hydrogen is evolved). Cool the mixture to ambient temperature and add 4 mL (51 mmol) of iodoethane. Heat the mixture again to 80° C. for 2 hours. Hydrolyse the reaction medium. Extract with dichloromethane, dry the organic phase over anhydrous sodium sulfate and evaporate. Purify the evaporation residue by chromatography on silica gel with elution by dichloromethane (Int. 49).

Yield: 89%

Yellow oil

1b) 5-Bromo-3-(2,4-dichlorobenzoyl)-1-ethyl-1H-indole

In 25 mL of dichloromethane, place 0.71 g (5.4 mmol) of aluminium chloride and 1.13 g (5.4 mmol) of 2,4-dichlorobenzoyl chloride. Stir for 2 hours and add to the reaction mixture 1.0 g (4.5 mmol) of 5-bromo-1-ethyl-1H-indole. Bring to reflux for 5 hours. Add water. Neutralize with a saturated solution of potassium carbonate. Extract with diethyl ether, dry over anhydrous sodium sulfate and evaporate. Take up the residue in a diisopropyl ether/methanol mixture, triturate in the cold and filter off the solid formed (Int. 50).

Yield: 91%

Melting point: 147-149° C. (diisopropyl ether/methanol)

TABLE 6

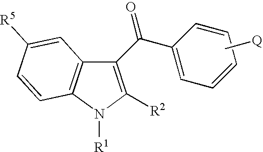

| N° | | | | | Yld | | Exam- |
|---|---|---|---|---|---|---|---|
| Int. | R¹ | R² | Q | R⁵ | (%) | Mp (° C.) | ple |
| 39 | H | H | 3-Cl | H | 31 | 235 | 13 |
| | | | | | | (diisopropyl ether) | |
| 40 | C₂H₅ | H | 3-Cl | H | 97 | oil | 13 |
| 43 | H | H | 2,4-diCl | H | 23 | 168 | 13 |
| | | | | | | (diisopropyl ether) | |
| 44 | C₂H₅ | H | 2,4-diCl | H | 96 | oil | 13 |
| 45 | H | CH₃ | 4-F | H | 22 | 197.5 | 13 |
| | | | | | | (diisopropyl ether) | |
| 46 | C₂H₅ | CH₃ | 4-F | H | 90 | oil | 13 |
| 47 | H | H | 3-Cl | Br | 57 | 240 | 14 |
| | | | | | | (diisopropyl ether/ | |
| | | | | | | absolute ethanol) | |
| 48 | C₂H₅ | H | 3-Cl | Br | 74 | 115 | 14 |
| | | | | | | (diisopropyl ether) | |

### Example 16

### 5-(4-Chlorobenzoyl)-1-ethyl-1H-indole

1a) 5-(4-Chlorobenzoyl)-1H-indole In a three-necked flask fitted with a coolant, a calcium chloride trap, a dropping funnel and under nitrogen, place 1.82 g (9.1 mmol) of potassium hydride in 20 mL of anhydrous tetrahydrofuran. Cool to 0° C. then add drop by drop 5-bromo-1H-indole diluted in 5 mL of tetrahydrofuran. After 15 minutes, the mixture is cooled to −78° C. and 12.2 mL (18.2 ml) of tert-butyllithium previously cooled to −78° C. are added using a dropper. After 15 minutes, 3.63 g (18.2 mmol) of N-methoxy-N-methyl-4-chlorobenzamide diluted in 5 mL of anhydrous tetrahydrofuran are added drop by drop. Allow to return slowly to ambient temperature then pour the solution slowly into 100 mL of 1M phosphoric acid previously cooled in ice; extract with diethyl ether, wash with an aqueous 5% solution of sodium bicarbonate, dry over anhydrous sodium sulfate and evaporate. Purify the evaporation residue by chromatography on silica gel with elution by diisopropyl ether (Int. 53).

Yield: 28%

Melting point: 130-132° C. (diisopropyl ether)

1b) 5-(4-Chlorobenzoyl)-1-ethyl-1H-indole

In 15 mL of anhydrous dimethylformamide, place 0.52 g (12.9 mmol) of sodium hydride in 60% suspension in mineral oil. Stir and heat the mixture to a temperature of 35° C. Add progressively, using a spatula, 1.1 g (4.3 mmol) of 5-(4-chlorobenzoyl)-1H-indole. Continue the heating for 30 minutes (until no further hydrogen is evolved). Cool the mixture to ambient temperature and add 0.7 mL (8.6 mmol) of iodoethane. Heat the mixture again to 35° C. for 1 hour. Add water, extract with dichloromethane, dry the organic phase over anhydrous sodium sulfate and evaporate. Purify the evaporation residue by chromatography on silica gel with elution by dichloromethane (Int. 54).

Yield: 77%

Melting point: 76-78° C. (diisopropyl ether)

### Example 17

### 5-Bromo-1-ethyl-7-(4-fluorobenzoyl)-1H-indole

1a) 7-(4-Fluorobenzoyl)-1H-indoline

In 50 mL of distilled toluene, introduce 2.5 g (21 mmol) of indoline and 3.05 g (25 mmol) of 4-fluorobenzonitrile. Cool to 5° C. and add progressively 2.7 g (23 mmol) of boron trichloride. Then add progressively 3.3 g (23 mmol) of aluminium chloride. Bring to reflux for 2.5 hours. Cool then add, at 8° C., 50 ml of water then 150 mL of 1M hydrochloric acid. Bring to reflux for 2 hours. Cool, extract the reaction medium with dichloromethane and evaporate the organic phase. Take up the evaporation residue in 200 mL of 10% w/v sodium hydroxide and stir for 1 hour. Extract with dichloromethane and wash the organic phase with water. Recover the organic phase, dry it over anhydrous sodium sulfate and evaporate. Isolate an orange powder by trituration in absolute ethanol (Int. 60).

Yield: 59%

Melting point: 131-133° C. (absolute ethanol)

1b) 1-Ethyl-7-(4-fluorobenzoyl)-1H-indoline

In 10 mL of anhydrous dimethylformamide, place 0.25 g (6.3 mmol) of sodium hydride in 60% suspension in mineral oil. Stir and heat the mixture to a temperature of 35° C. Add progressively, using a spatula, 0.5 g (2.1 mmol) of 7-(4-fluorobenzoyl)-1H-indoline. Continue the heating for 30 minutes (until no further hydrogen is evolved). Cool the mixture to ambient temperature and add 0.25 mL (3.15 mmol) of iodoethane. Heat the mixture again to 35° C. for 1 hour. Add water, extract with dichloromethane, dry the organic phase over anhydrous sodium sulfate and evaporate. Purify the evaporation residue by chromatography on silica gel with elution by dichloromethane (Int. 61).

Yield: 56%

Yellow oil

1c) 5-Bromo-7-(4-fluorobenzoyl)-1H-indoline In 100 mL of dichloromethane, introduce 1 g (4.1 mmol) of 7-(4-fluorobenzoyl)-1H-indoline. Add 0.81 g (4.6 mmol) of N-bromosuccinimide and stir for 12 hours at ambient temperature. Wash the organic phase successively with water and a saturated solution of sodium bicarbonate. Dry the organic phase over anhydrous sodium sulfate and evaporate the solvent. Purify the evaporation residue by chromatography on a silica gel column with elution by dichloromethane. Isolate a bright yellow powder (Int. 62).

Yield: 75%

Melting point: 124-126° C. (petroleum ether)

TABLE 7

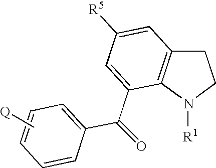

| N° Int. | R¹ | Q | R⁵ | Yld (%) | Mp (° C.) |
|---|---|---|---|---|---|
| 55 | H | 4-Br | H | 61 | 124-126 |
| | | | | | (petroleum |
| | | | | | ether) |
| 56 | H | 3-Cl | H | 62 | 89-90 |
| | | | | | (methanol) |
| 57 | C₂H₅ | 3-Cl | H | 53 | oil |
| 58 | H | 4-Cl | H | 65 | 109-111 |
| | | | | | (petroleum |
| | | | | | ether) |
| 59 | C₂H₅ | 4-Cl | H | 55 | oil |
| 63 | C₂H₅ | 4-F | Br | 68 | oil |
| 64 | H | 4-F | Cl | 69 | 138-140 |
| | | | | | (dichloro- |
| | | | | | methane) |
| 65 | C₂H₅ | 4-F | Cl | 80 | 135-135 |
| | | | | | (petroleum |
| | | | | | ether) |

1d) 5-Bromo-7-(4-fluorobenzoyl)-1H-indole

In 20 mL of dichloromethane, introduce 0.5 g (1.6 mmol) of 5-bromo-7-(4-fluorobenzoyl)-1H-indoline and 0.82 g (9.43 mmol) of manganese oxide. Filter over celite and evaporate the solvent (Int. 74).

Yield: 90%

Melting point: 123-125° C. (dichloromethane)

1e) 5-Bromo-1-ethyl-7-(4-fluorobenzoyl)-1H-indole

In 10 mL of anhydrous dimethylformamide, place 0.15 g (3.69 mmol) of sodium hydride in 60% suspension in mineral oil. Stir and heat the mixture to a temperature of 35° C. Add progressively, using a spatula, 0.4 g (1.26 mmol) of 5-bromo-7-(4-fluorobenzoyl)-1H-indole. Continue the heating for 30 minutes (until no further hydrogen is evolved). Cool the mixture to ambient temperature and add 0.15 mL (1.89 mmol) of iodoethane. Heat the mixture again to 35° C. for 1 hour. Add water, extract with dichloromethane, dry the organic phase over anhydrous sodium sulfate and evaporate. Purify the evaporation residue by chromatography on silica gel with elution by dichloromethane (Int. 75).

Yield: 83%

Yellow oil

TABLE 8

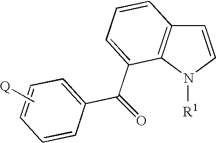

| N° Int. | R¹ | Q | Yld (%) | Mp (° C.) |
|---|---|---|---|---|
| 66 | H | 4-Br | 92 | 170-173 |
| | | | | (petroleum ether) |
| 67 | C₂H₅ | 4-Br | 52 | oil |
| 68 | H | 3-Cl | 86 | 84-85 |
| | | | | (petroleum ether) |
| 69 | C₂H₅ | 3-Cl | 91 | 68-70 |
| | | | | (dichloromethane) |
| 70 | H | 4-Cl | 90 | 155-157 |
| | | | | (petroleum ether) |
| 71 | C₂H₅ | 4-Cl | 63 | oil |
| 72 | H | 4-F | 96 | 110-113 |
| | | | | (diisopropyl ether) |
| 73 | C₂H₅ | 4-F | 77 | oil |

II.2. Preparation of the Final Compounds

### Example 18

### 1-Ethyl-2-[(4-fluorophenyl)(1H-imidazol-1-yl)methyl]-3-methyl-1H-indole

According to Example 10: Cpsé 31

Yield: 47%

Yellow oil

### Example 19

### 5-Bromo-1-ethyl-2-[(4-fluorophenyl)(1H-1,2,4-triazol-1-yl)methyl]-3-methyl-1H-indole

In 10 mL of methanol, introduce 0.17 g (4.5 mmol) of sodium borohydride. Stir the mixture and add progressively, using a dropping funnel, a solution of 0.55 g (1.5 mmol) of 5-bromo-1-ethyl-2-(4-fluorobenzoyl)-3-methyl-1H-indole in 5 mL of methanol. Stir at ambient temperature for 1 hour. Add water and extract with diethyl ether. Dry the organic phase over anhydrous sodium sulfate and evaporate.

In 20 mL of acetonitrile, introduce 2.42 g (35 mmol) of 1,2,4-1H-triazole. Cool the solution in an ice bath. Add progressively, using a dropping funnel, 0.64 mL (8.8 mmol) of thionyl chloride. Stir at ambient temperature for 1 hour. Filter and cool the filtrate in an ice bath. Add progressively, using a dropping funnel, a solution of 0.80 g (2.2 mmol) of 5-bromo-1-ethyl-2-[(4-fluorophenyl)(hydroxy)methyl]-3-methyl-1H-indole in 7 mL of acetonitrile. Stir at 0° C. for 1 hour then at ambient temperature for 1 hour. Filter and evaporate the solvent. Take up the residue in water, extract with dichloromethane, dry the organic phase over anhydrous sodium sulfate and evaporate. Purify the evaporation residue by chromatography on silica gel with elution by a 19/1 dichloromethane/absolute ethanol mixture. Take up the oily residue in methanol and triturate in the cold. Filter the triazole (Cpsé 39).

Yield: 68%

Melting point: 138-139 (methanol)

TABLE 9

| | |
|---|---|
| | (IB2) |
| 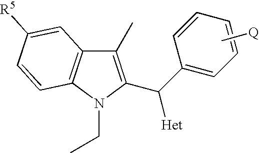 | |

| N° | | | | Derv | Yld | | Exam- |
|---|---|---|---|---|---|---|---|
| Cpsé | Het | Q | R⁵ | from | (%) | Mp (° C.) | ple |
| 26 | Imid | 3-Br | H | 29 | 46 | oil | 10 |
| 27 | Imid | 4-Br | H | 30 | 28 | oil | 10 |
| 28 | Imid | 3-Cl | H | 31 | 71 | oil | 10 |
| 29 | Imid | 4-Cl | H | 32 | 68 | oil | 10 |
| 30 | Imid | 2,4-diCl | H | 33 | 80 | 187-189 | 10 |
| | | | | | | (diethyl ether) | |
| 32 | Imid | 3-Cl | Br | 35 | 57 | 143-144 | 10 |
| | | | | | | (ethyl acetate) | |
| 33 | Imid | 4-Cl | Br | 36 | 53 | 86-88 | 10 |
| | | | | | | (methanol) | |
| 34 | Imid | 2,4-diCl | Br | 37 | 66 | 173-174 | 10 |
| | | | | | | (diisopropyl | |
| | | | | | | ether) | |
| 35 | 1-Triazol | 3-Cl | Br | 35 | 40 | 73-75 | 19 |
| | | | | | | (acetonitrile) | |
| 36 | 1-Triazol | 4-Cl | Br | 36 | 67 | 73-75 | 19 |
| | | | | | | (methanol) | |
| 37 | 1-Triazol | 2,4-diCl | Br | 37 | 46 | 136-137 | 19 |
| | | | | | | (acetonitrile) | |
| 38 | 4-Triazol | 2,4-diCl | Br | 37 | 16 | oil | 19 |

### Example 20

### 5-Bromo-1-ethyl-3-[(4-fluorophenyl)(1H-imidazol-1yl)methyl]-1H-indole

According to Example 10: Cpsé 46

Yield: 71%

Melting point: 143-145° C. (diisopropyl ether)

### Example 21

### 5-Bromo-1-ethyl-3-[(2,4-dichlorophenyl)(1H-1,2,4-triazol-1yl)methyl]-1H-indole

According to Example 19: Cpsé 47

Yield: 30%

Melting point: 124-126° C. (acetonitrile)

TABLE 10

| | |
|---|---|
| | (IB3) |
| 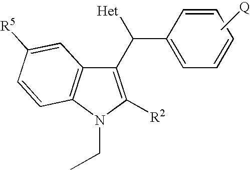 | |

| N° | | | | | Derv | Yld | | Exam- |
|---|---|---|---|---|---|---|---|---|
| Cpsé | Het | R² | Q | R⁵ | from | (%) | Mp (° C.) | ple |
| 40 | Imid | H | 3-Cl | H | 40 | 45 | oil | 10 |
| 41 | Imid | H | 4-Cl | H | 42 | 55 | oil | 10 |
| 42 | Imid | H | 2,4-diCl | H | 44 | 58 | oil | 10 |
| 43 | Imid | CH₃ | 4-F | H | 46 | 52 | oil | 10 |
| 44 | Imid | H | 3-Cl | Br | 48 | 63 | 163 | 10 |
| | | | | | | | (diisopropyl | |
| | | | | | | | ether) | |
| 45 | Imid | H | 2,4-diCl | Br | 50 | 66 | 138-140 | 10 |
| | | | | | | | (diisopropyl | |
| | | | | | | | ether) | |

### Example 22

### 5-Bromo-1-ethyl-7-[(4-fluorophenyl)(1H-imidazol-1-yl)methyl]indoline

According to Example 10: Cpsé 51

Yield: 30%

Melting point: 120-123° C. (petroleum ether)

TABLE 11

| | |
|---|---|
| | (IB4) |
| 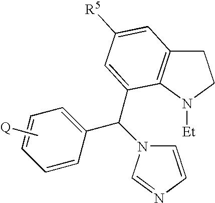 | |

| N° Cpsé | Q | R⁵ | Derv from | Yld (%) | Mp (° C.) | Example |
|---|---|---|---|---|---|---|
| 48 | 3-Cl | H | 57 | 55 | 118-120 | 10 |
| | | | | | (petroleum | |
| | | | | | ether) | |
| 49 | 4-Cl | H | 59 | 58 | oil | 10 |
| 50 | 4-F | H | 61 | 50 | 103-104 | 10 |
| | | | | | (petroleum | |
| | | | | | ether) | |
| 52 | 4-F | Cl | 65 | 49 | 120-122 | 10 |
| | | | | | (petroleum | |
| | | | | | ether) | |

### Example 23

### 5-[(4-Chlorophenyl)(1H-imidazol-1-yl)methyl]-1-ethyl-1H-indole

According to Example 10: Cpsé 53

Yield: 30%

yellow oil

TABLE 12

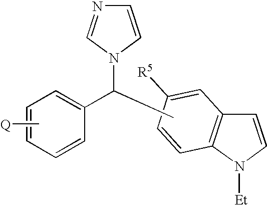

| N° | | | Site of | Derv | Yld | | |
|---|---|---|---|---|---|---|---|
| Cpsé | Q | R⁵ | fixation | from | (%) | Mp (° C.) | Example |
| 54 | 4-Br | H | 7 | 67 | 45 | 145 | 10 |
| | | | | | | (decomposition) | |
| 55 | 3-Cl | H | 7 | 69 | 65 | 150 | 10 |
| | | | | | | (decomposition) | |
| 56 | 4-Cl | H | 7 | 71 | 70 | 120 | 10 |
| | | | | | | (decomposition) | |
| 57 | 4-F | H | 7 | 73 | 49 | 97-99 | 10 |
| | | | | | | (petroleum ether) | |
| 58 | 4-F | Br | 7 | 75 | 20 | 121-123 | 10 |
| | | | | | | (petroleum ether) | |

### Synthesis of Compounds of Formula (ID)

1. Preparation of the Intermediates

### Example 24

### 2-[(4-Chlorobenzyl)-1H-indol-3-yl]ethyl Acetate

In 30 mL of anhydrous dimethyl sulfoxide, place 0.51 g (21 mmol) of sodium hydride in 60% suspension in mineral oil. Stir and heat the mixture to a temperature of 35° C. Add progressively, using a spatula, 4.27 g (21 mmol) of 1H-indol-3-ylethyl acetate. Continue the heating for 30 minutes (until no further hydrogen is evolved). Cool the mixture to ambient temperature and add 3.38 g (21 mmol) of 4-chlorobenzyl chloride. Heat the mixture again to 35° C. for 1 hour. Add water, extract with dichloromethane, dry the organic phase over anhydrous sodium sulfate and evaporate (Int. 76).

Yield: 35%

Melting point: 84-86° C. (diisopropyl ether)

TABLE 13

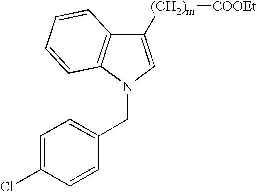

| N° Int. | m | Yld (%) | Mp (° C.) |
|---|---|---|---|
| 77 | 2 | 51 | oil |
| 78 | 3 | 30 | 111-112 |
| | | | (diisopropyl |
| | | | ether) |

### Example 25

### 1-[1-(4-Chlorobenzyl)-1H-indol-3-yl]-1-formylcyclopentane

1a) [1-(4-chlorobenzyl)-1H-indol-3-yl]acetonitrile

In 20 mL of anhydrous dimethylsulfoxide, place 0.38 g (9.6 mmol) of sodium hydride in 60% suspension in mineral oil. Stir and heat the mixture to a temperature of 35° C. Add progressively, using a spatula, 1.5 g (9.6 mmol) of 1H-indol-3-ylacetonitrile. Continue the heating for 30 minutes (until no further hydrogen is evolved). Cool the mixture to ambient temperature and add 1.85 g (11.52 mmol) of 4-chlorobenzyl chloride. Heat the mixture again to 35° C. for 1 hour. Add water, extract with dichloromethane, dry the organic phase over anhydrous sodium sulfate and evaporate (Int. 82).

Yield: 70%

Melting point: 84-85° C. (diisopropyl ether)

1b) 1-[1-(4-Chlorobenzyl)-1H-indol-3-yl]-1-cyanocyclopentane

In 20 mL of anhydrous dimethylsulfoxide, place 0.30 g (7.48 mmol) of sodium hydride in 60% suspension in mineral oil. Stir and heat the mixture to a temperature of 35° C. Add progressively, using a spatula, 1.0 g (3.56 mmol) of 1-(4-chlorobenzyl)-1H-indol-3-ylacetonitrile. Continue the heating for 30 (until no further hydrogen is evolved). Cool the mixture to ambient temperature and add 0.85 g (3.92 mmol) of 1,4-dibromobutane. Heat the mixture again to 35° C. for 1 hour. Add water, extract with dichloromethane, dry the organic phase over anhydrous sodium sulfate and evaporate (Int. 83).

Yield: 74%

Melting point: 99-101° C. (diisopropyl ether)

1c) 1-[1-(4-Chlorobenzyl)-1H-indol-3-yl]-1-formylcyclopentane

Under nitrogen atmosphere, add 1 g (2.99 mmol) of 1-[1-(4-chlorobenzyl)-1H-indol-3-yl]-1-cyanocyclopentane in 20 mL of toluene previously cooled to −60° C. Stir and add progressively, using a dropping funnel, 4.78 mL (4.78 mmol) of an n-hexane solution of DIBAH. Stir and let the temperature rise progressively to ambient, over 3 hours. Add 1.5 mL of methanol and 31 mL of 1M hydrochloric acid to the mixture. Extract with dichloromethane, dry the organic phase over anhydrous sodium sulfate and filter. Evaporate and triturate the residue in the cold in diisopropyl ether (Int. 84).

Yield: 79%

Melting point: 148-150° C. (diisopropyl ether)

TABLE 14
Intermediates according to example 25

| N° Int. | Structure | Yld (%) | Mp (° C.) |
|---|---|---|---|
| 79 | 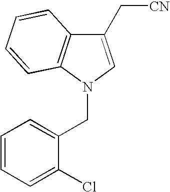 | 62 | 109-110 (diisopropyl ether) |
| 80 | 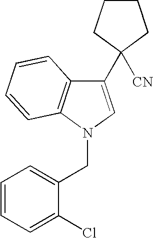 | 53 | 123-125 (diisopropyl ether) |
| 81 | 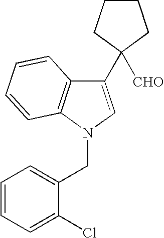 | 72 | 155-156 (diisopropyl ether) |

### Example 26

### 1-(2,4-Dichlorobenzyl)-3-(1-hydroxyethyl)-2-methyl-1H-indole

Under nitrogen atmosphere, cool a solution of 0.35 g (1.1 mmol) of 1-(2,4-dichlorobenzyl)-3-formyl-2-methyl-1H-indole in 15 mL of anhydrous tetrahydrofuran at −78° C. Add, drop by drop, 0.4 mL (1.21 mmol) of methylmagnesium chloride (3.0 M in tetrahydrofuran). Stir the mixture for 30 minutes. Add a saturated solution of ammonium chloride and wash with a saturated aqueous solution of sodium bicarbonate and a saturated aqueous solution of sodium chloride. Dry the organic phase over anhydrous sodium sulfate. Evaporate the solvent and triturate in the cold in diisopropyl ether. Filter off the solid (Int. 92).

Yield: 84%

Melting point: 107-108° C. (diisopropyl ether)

TABLE 15
Intermediates according to example 26

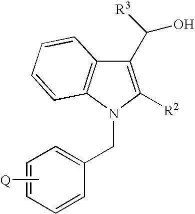

| N° Int. | R² | R³ | Q | Yld (%) | Mp (° C.) |
|---|---|---|---|---|---|
| 85 | H | ethyl | 4-Cl | quantitative | oil |
| 86 | H | n-propyl | 4-Cl | quantitative | oil |
| 87 | H | n-butyl | 4-Cl | quantitative | oil |
| 88 | H | methyl | 4-F | quantitative | oil |
| 89 | H | methyl | 2,4-diCl | quantitative | oil |
| 90 | H | methyl | 2,4-diF | quantitative | oil |
| 91 | CH₃ | methyl | 4-Br | quantitative | oil |
| 93 | CH₃ | methyl | 4-F | quantitative | oil |

2. Preparation of the Final Compounds

### Example 27

### 1-(4-Chlorobenzyl)-3-(2-1H-imidazol-1-ylethyl)-1H-indole

According to Example 9: Cpsé 59

Yield: 84%

Melting point: 78-80° C. (diisopropyl ether)

TABLE 16

| | |
|---|---|
| | (ID1) |
| 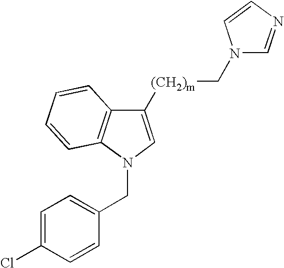 | |

| N° Cpsé | m | Derv from | Yld (%) | Mp (° C.) |
|---|---|---|---|---|
| 60 | 2 | 77 | 78 | 65-67 |
| | | | | (diisopropyl |
| | | | | ether) |
| 61 | 3 | 78 | 91 | oil |

### Example 28

### 1-[1-(4-Chlorobenzyl)-1H-indol-3yl]-1-(1H-imidazol-1-ylmethyl)cyclopentane

According to Example 10: Cpsé 63

Yield: 70%

Melting point: 91-92° C. (ethanol)

TABLE 17

| | |
|---|---|
| | (ID2) |
| 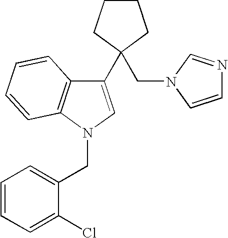 | |

| N° Cpsé | Derv from | Yld (%) | Mp (° C.) |
|---|---|---|---|
| 62 | 81 | 73 | oil |

### Example 29

### 1-(2,4-Dichlorobenzyl)-3-[(1H-imidazol-1-yl)(methyl)methyl]-2-methyl-1H-indole

In 20 mL of anhydrous tetrahydrofuran, introduce 0.27 g (0.81 mmol) of 1-(2,4-dichlorobenzyl-3-(1-hydroxyethyl)-2-methyl-1H-indole and 0.13 g (0.81 mmol) of 1,1′-carbonyldiimidazole. Stir and bring to reflux for 4 hours. Concentrate the solution and take up the residue in water. Extract with dichloromethane, dry the organic phase over anhydrous sodium sulfate and evaporate. Purify the evaporation residue by chromatography on silica gel with elution by a 19/1 dichloromethane/absolute ethanol mixture: (Cpsé 71).

Yield: 63%

Green oil

TABLE 18

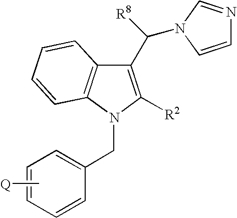

| N° | | | | | | |
|---|---|---|---|---|---|---|
| Cpsé | R² | R⁸ | Q | Derv from | Yld (%) | Mp (° C.) |
| 64 | H | ethyl | 4-Cl | 85 | 64 | oil |
| 65 | H | n-propyl | 4-Cl | 86 | 53 | oil |
| 66 | H | n-butyl | 4-Cl | 87 | 47 | oil |
| 67 | H | methyl | 4-F | 88 | 68 | oil |
| 68 | H | methyl | 2,4-diCl | 89 | 79 | oil |
| 69 | H | methyl | 2,4-diF | 90 | 62 | oil |
| 70 | CH₃ | methyl | 4-Br | 91 | 54 | oil |
| 72 | CH₃ | methyl | 4-F | 93 | 45 | 137-140 |
| | | | | | | (diisopropyl |
| | | | | | | ether) |

### Synthesis of Compounds of Formulas (IE) and (IF)

1. Preparation of the Intermediates

### Example 30

### 1-Hydroxymethyl-1H-benzotriazole

Dissolve 10 g (83.94 mmol) of 1H-benzotriazole in 6.81 mL (83.94 mmol) of a 37% aqueous solution of formaldehyde. Stir and bring the mixture to a temperature of 25° C. After 5 minutes, the reaction mixture solidifies. Cool the solution to ambient temperature. Filter and wash with diethyl ether. Triturate the residue in the cold in tetrahydrofuran and filter (Int. 94).

Yield: 94%

Melting point: 148-150° C. (tetrahydrofuran and diethyl ether)

### Example 31

### 1-Chloromethyl-1H-benzotriazole

To 8.91 g (59.7 mmol) of 1-hydroxymethyl-1H-benzotriazole cooled to 0° C. in an ice bath, add, progressively, using a dropping funnel, 26 mL (360 mmol) of thionyl chloride. Stir and bring to reflux for 1 hour. Evaporate to dryness. Take up the residue in methanol. Cool the solution, filter and dry (Int. 95).

Yield: 93%

Melting point: 136-138° C. (methanol)

### Example 32

### 1-(1H-Benzotriazol-1-ylmethyl)-1H-indole

In 30 mL of anhydrous dimethyl sulfoxide, introduce 1.21 g (30.32 mmol) of sodium hydride in 60% suspension in a mineral oil. Stir at ambient temperature, and add progressively 3.23 g (27.6 mmol) of indole. Continue the stirring for 1 hour at ambient temperature (until no further hydrogen is evolved). Introduce progressively 4.62 g (27.56 mmol) of 1-chloromethyl-1H-benzotriazole. Stir the mixture again for 2 hours. Add water. Extract with dichloromethane, wash with water and dry the organic phase over anhydrous sodium sulfate. Filter and evaporate to dryness. Triturate the oily residue in the cold in diisopropyl ether and filter (Int. 96).

Yield: 80%

Melting point: 176-178° C. (diisopropyl ether)

### Example 33

### 1-[(1H-Benzotriazol-1-yl)(4-bromobenzoyl)methyl]-1H-indole

Cool a solution of 1.88 g (7.57 mmol) of N-(1H-benzotriazol-1-ylmethyl)-1H-indole in 50 mL of tetrahydrofuran at −78° C. Add, drop by drop, 5.67 mL (9.08 mmol) of n-butyllithium (1.6 M in tetrahydrofuran). Stir the mixture for 1 hour; the solution turns dark brown. Add, drop by drop, 2.17 g (9.46 mmol) of ethyl 4-bromobenzoate; the solution clears, becoming light brown. Allow to return to ambient temperature for 12 hours. Add a saturated solution of ammonium chloride and add 30 mL of water to dissolve the precipitate formed (lithium chloride). Extract with diethyl ether, wash with water and dry the organic phase over anhydrous sodium sulfate. Evaporate the solvent and purify the solid obtained by chromatography on a silica gel column with elution by dichloromethane. Evaporate the solvent. Triturate in the cold in diisopropyl ether. Filter off the solid (Int. 97).

Yield: 50%

Melting point: 156-157° C. (diisopropyl ether)

TABLE 19
Intermediates according to example 33

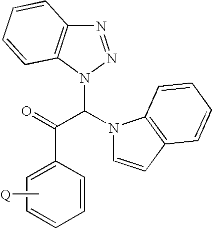

| N° Int. | Q | Yld (%) | Mp (° C.) |
|---|---|---|---|
| 98 | 4-Cl | 61 | 184-185 |
| | | | (diisopropyl ether) |
| 99 | 2,4-diCl | 51 | 141-142 |
| | | | (diisopropyl ether) |
| 100 | 4-F | 61 | 121-123 |
| | | | (diisopropyl ether) |
| 101 | 2,4-diE | 41 | 111-113 |
| | | | (diisopropyl ether) |
| 102 | 4-CF3 | 75 | 175-179 |
| | | | (diisopropyl ether) |

### Example 34

### 1-(4-Bromobenzoylmethyl)-1H-indole

In a solution of 1.3 g (3.00 mmol) of 1-[(1H-benzotriazol-1-yl)(4-bromobenzoyl) methyl]-1H-indole in 15 mL absolute ethanol and 15 mL of tetrahydrofuran, add 3 mL of acetic acid. Stir and add 0.98 g (15 mmol) of zinc. Stir the mixture in an ultrasound bath for 5 hours at 30-40° C. Filter over Celite 545 and evaporate the filtrate. Extract with dichloromethane, wash with water and dry the organic phase over anhydrous sodium sulfate. Evaporate the solvent and take up the residue in a ethanol/diisopropyl ether mixture. Filter off the solid (Int. 103).

Yield: 66%

Melting point: 185-186° C. (diisopropyl ether)

TABLE 20
Intermediates according to example 34

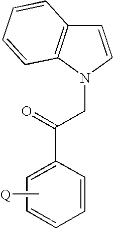

| N° Int. | Q | Yld (%) | Mp (° C.) |
|---|---|---|---|
| 104 | 4-Cl | 66 | 156-157 |
| | | | (diisopropyl ether) |
| 105 | 2,4-diCl | 46 | 98-99 |
| | | | (diisopropyl ether) |
| 106 | 4-F | 57 | 153-154 |
| | | | (diisopropyl ether) |
| 107 | 2,4-diF | 60 | 101-103 |
| | | | (diisopropyl ether) |
| 108 | 4-CF₃ | 45 | 182-185 |
| | | | (diisopropyl ether |
| | | | and dichloromethane) |

### Example 35

### 1-(Ethoxycarbonyl-2ethyl)-1H-indole

In 10 ml of anhydrous dimethyl sulfoxide, place 1 g (24.5 mmol) of sodium hydride in 60% suspension in mineral oil. Add progressively, using a spatula, 2.34 g (20 mmol) of 1H-indole at ambient temperature. Stir for 30 minutes and add, drop by drop, 20 ml of a solution of 4.45 g (24.5 mmol) of ethyl bromopropionate in dimethyl sulfoxide. Heat the mixture again to 30° C. for 5 hours. Add water, extract with dichloromethane, dry the organic phase over anhydrous sodium sulfate and evaporate. Purify the evaporation residue by chromatography on silica gel with elution by a 1/1 dichloromethane/n-hexane mixture: (Int. 109).

Yield: 85%

Yellow oil

### Example 36

### 1-(2,4-Difluorobenzoyl-2ethyl)-1H-indole

Under a nitrogen atmosphere, cool a solution of 2 g (10.36 mmol) of bromo-2,4-difluorobenzene in 10 ml of tetrahydrofuran at −78° C. Add, drop by drop, 4.1 ml (10.36 mmol) of n-butyllithium (2.5 M in hexane). Stir the mixture for 40 minutes. The solution turns dark brown. Add, drop by drop, 2.25 g (10.36 mmol) of 1-(ethoxycarbonyl-2-ethyl)-1H-indole. Stir the mixture for 1 hour at the same temperature and allow to return to ambient temperature for 1 hour. Add a saturated solution of ammonium chloride and add 30 ml of water to dissolve the solids formed. Extract with ethyl acetate, wash with water and dry the organic phase over anhydrous sodium sulfate and evaporate. Purify the evaporation residue by chromatography on silica gel with elution by a 1/1 dichloromethane/n-hexane mixture (Int. 110).

Yield: 50%

Yellow oil

TABLE 21
Intermediate according to example 36

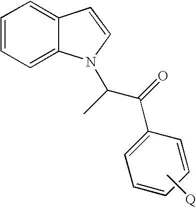

| N^{o} Int. | Q | Yld (%) | Mp (° C.) |
|---|---|---|---|
| 111 | 2,4-diCl | 20 | oil |

### Example 37

### 2-(4-Bromophenyl)-3-(indol-1-yl)-1,2-epoxypropane

Under a nitrogen atmosphere, to a suspension of 0.42 g (1.90 mmol) of trimethylsulfoxonium iodide in 3 mL of dimethyl sulfoxide, add 69.50 mg (1.74 mmol) of sodium hydride at 10° C. Stir at 10° C. for 30 minutes, allow to return to ambient temperature for 30 minutes. Add a solution of 0.52 g (1.66 mmol) of N-(4-bromobenzoylmethyl)-1H-indole in 10 mL of anhydrous tetrahydrofuran and stir at ambient temperature for 50 hours. Extract with ethyl acetate, wash with water and dry the organic phase over anhydrous sodium sulfate. Evaporate the solvent and purify the residue obtained by chromatography on a silica gel column with elution by a 1/1 dichloromethane/hexane mixture. Evaporate the solvent (Int. 112).

Yield: 37%

Clear oil

TABLE 22
Intermediates according to example 37

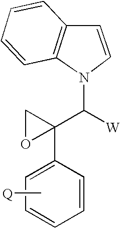

| N^{o} Int. | W | Q | Yld (%) | Mp (° C.) |
|---|---|---|---|---|
| 114 | H | 2,4-diCl | 61 | oil |
| 118 | CH₃ | 2,4-diCl | 70 | oil |

### Example 38

### 2-(4-Chlorophenyl)-3-(indol-1-yl)-1,2-epoxypropane

To a solution of 0.38 g (1.41 mmol) of N-(4-chlorobenzoylmethyl)-1H-indole in 5 mL of dichloromethane, add 0.44 g (2.02 mmol) of trimethylsulfoxonium iodide and an aqueous solution of 6 g (150 mmol) of 48% sodium hydroxide. Stir at ambient temperature for 48 hours.

Add water, extract with dichloromethane and dry the organic phase over anhydrous sodium sulfate. Evaporate the solvent and purify the residue obtained by chromatography on a silica gel column with elution by a 1/1 dichloromethane/hexane mixture. Evaporate the solvent (Int. 113).

Yield: 75%

Clear oil

TABLE 23
Intermediates according to example 38

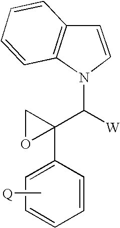

| N^{o} Int. | W | Q | Yld (%) | Mp (° C.) |
|---|---|---|---|---|
| 115 | H | 4-F | 40 | oil |
| 116 | H | 2,4-diF | 59 | oil |
| 117 | H | 4-CF₃ | 62 | oil |
| 119 | CH₃ | 2,4-diF | 30 | oil |

### Example 39

### 2-(1H-Benzotriazol-1-yl)-1-(4-fluorophenyl)-2-(indol-1-yl)ethanol

Cool a solution of 1.88 g (7.57 mmol) of 1-(1H-benzotriazol-1-ylmethyl)-1H-indole in 50 mL of tetrahydrofuran at −78° C. Add, drop by drop, 3.63 mL (5.80 mmol) of n-butyllithium (1.6 M in tetrahydrofuran). Stir the mixture for 1 hour; the solution turns dark brown. Add, drop by drop, 1.86 g (10.06 mmol) of 4-bromobenzaldehyde. Allow to return to ambient temperature for 12 hours. Add a saturated solution of ammonium chloride and add 30 mL of water to dissolve the solids (lithium chloride). Extract with diethyl ether, wash with water and dry the organic phase over anhydrous sodium sulfate. Evaporate the solvent and purify the solid obtained by chromatography on a silica gel column with elution by dichloromethane. Evaporate the solvent. Triturate in the cold in diisopropyl ether. Filter off the solid (Int. 120).

Yield: 48%

Melting point: 206-207° C. (diisopropyl ether)

### Example 40

### 3-Methyl-1-tosyl-1H-indole

In 100 mL of anhydrous dimethyl sulfoxide, introduce 2.24 g (56.0 mmol) of sodium hydride in 60% suspension in mineral oil. Stir at ambient temperature, and add progressively, using a spatula, 7 g (53.4 mmol) of 3-methylindole. Continue the stirring for 1 hour (until no further hydrogen is evolved). Add 10.68 g (53.4 mmol) of p-toluenesulfonyl chloride. Stir the mixture again for 2 hours. Add water. Extract with dichloromethane, wash with water and dry the organic phase over anhydrous sodium sulfate. Evaporate to dryness. Triturate the oily residue in the cold in diisopropyl ether and filter (Int. 121).

Yield: 96%

Melting point: 99-101 (diisopropyl ether)

### Example 41

### 3-Bromomethyl-1-tosyl-1H-indole

In a 250 ml flask, under nitrogen atmosphere, add 3.77 g (13 mmol) of 1-tosyl-3-methyl-1H-indole in 124 ml of CCl_{4.} Then add 2.47 g (178 mmol) of N-bromosuccinimide and 124 mg of azobisisobutyronitrile progressively. Heat to reflux for 20 minutes. Then add 61.5 mg of azobisisobutyronitrile every 7 minutes for 21 minutes. Heat to reflux for 50 minutes, then cool. The precipitate is filtered off and washed with petroleum ether. The filtrate is evaporated (Int. 122).

Yield: 89%

Melting point: 143-145 (diethyl ether)

### Example 42

### 3-(2,4-Dichlorobenzoylmethyl)-1H-indol

In a 100 ml three-necked flask, add 1.4 g (3.8 mmol) of 1-tosyl-3-bromomethyl-1H-indole to a solution of 30 ml of anhydrous anisole. Heat to 80° C. under a carbon monoxide atmosphere. Then add 1.59 g (11.5 mmol) of K₂CO₃ and 0.73 g (3.8 mmol) of 2,4-dichlorobenzeneboronic acid. Stir for 5 hours. Extract with toluene, wash with water then with a saturated sodium chloride solution. Dry with ammonium sulfate (int. 123).

Yield: 91%

Brown oil

### Example 43

### 2-(2.4-Dichlorophenyl)-3-(1-tosylindol-3-yl)-1,2-epoxypropane

Under a nitrogen atmosphere, to a suspension of 1.4 g (6.38 mmol) of trimethylsulfoxonium iodide in 10 mL of dimethyl sulfoxide, add 0.153 g (3.83 mmol) of sodium hydride at 10° C. Stir at 10° C. for 30 minutes, allow to return to ambient temperature for 30 minutes. Add a solution of 1.27 g (3.19 mmol) of 3-(2,4-dichlorobenzoylmethyl)-1-tosyl-1H-indole in 10 mL of anhydrous tetrahydrofuran and stir at ambient temperature for 50 hours. Extract with ethyl acetate, wash with water and dry the organic phase over anhydrous sodium sulfate. Evaporate the solvent and purify the residue obtained by chromatography on a silica gel column with elution by a 1/1 dichloromethane/hexane mixture. Evaporate the solvent (Int. 124).

Yield: 33%

Yellow oil

2. Preparation of the Final Compounds

### Example 44

### 2-(4-Bromophenyl)-1-(1H-imidazol-1-yl)-3-(indol-1-yl)propan-2-ol

Under a nitrogen atmosphere, to a solution of 0.21 g (0.64 mmol) of 2-(4-bromophenyl)-3-indol-1-yl-1,2-epoxypropane in 20 mL of dimethylformamide, add 0.25 g (1.83 mmol) of potassium carbonate and 0.12 g (1.82 mmol) of 1H-imidazole. Heat to 90° C. for 7 hours. Extract with diethyl ether, wash with water and dry the organic phase over anhydrous sodium sulfate. Evaporate the solvent and purify the solid obtained by chromatography on a silica gel column with elution by a 19/1 dichloromethane/ethanol mixture. Evaporate the solvent. Triturate in the cold in diisopropyl ether. Filter off the solid (Cpsé 73).

Yield: 21%

Melting point: 134-135° C. (diisopropyl ether)

TABLE 24
Final compounds according to example 44

| | |
|---|---|
| | (IF) |
| 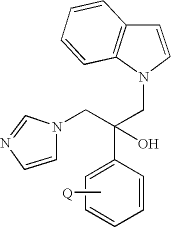 | |

| N^{o} Cpsé | Q | Derv from | Yld (%) | Mp (° C.) |
|---|---|---|---|---|
| 74 | 4-Cl | 113 | 60 | 201-202 |
| | | | | (diisopropyl ether) |
| 75 | 2,4-diCl | 114 | 70 | >250 |
| | | | | (diisopropyl ether) |
| 76 | 4-F | 115 | 50 | 166-167 |
| | | | | (diisopropyl ether) |
| 77 | 2,4-diF | 116 | 50 | 166-168 |
| | | | | (diisopropyl ether) |

### Example 45

### 2-(2,4-Dichlorophenyl)-1-(1,2,4-1H-triazol-1-yl)-3-(indol-1-yl)propan-2-ol

Under a nitrogen atmosphere, to a solution of 0.19 g (0.60 mmol) of 2-(2,4-dichlorophenyl)-3-indol-1-yl-1,2-epoxypropane in 20 mL of dimethylformamide, add 0.17 g (1.20 mmol) of potassium carbonate and 0.083 g (1.20 mmol) of 1,2,4-1H-triazole. Heat to 90° C. for 24 hours. Extract with diethyl ether, wash with water and dry the organic phase over anhydrous sodium sulfate. Evaporate the solvent and purify the solid obtained by chromatography on a silica gel column with elution by a 19/1 dichloromethane/ethanol mixture. Evaporate the solvent. Triturate in the cold in diisopropyl ether. Filter off the solid (Cpsé 78).

Yield: 47%

Melting point: 59-160° C. (diisopropyl ether)

TABLE 25
Final compounds according to example 45

| | |
|---|---|
| | (IF) |
| 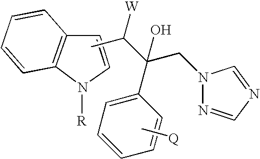 | |

| N^{o} | Site of | | | | Derv | Yld | |
|---|---|---|---|---|---|---|---|
| Cpsé | fixation | R | W | Q | from | (%) | Mp (° C.) |
| 79 | 1 | — | H | 4-CF₃ | 117 | 86 | oil |
| 80 | 1 | — | CH₃ | 2,4-Cl | 118 | 96 | oil |
| 81 | 1 | — | CH₃ | 2,4-diF | 119 | 10 | oil |
| 82 | 3 | tosyl | H | 2,4-diCl | 124 | 35 | oil |

### Example 46

### 1-[2-(4-Fluorophenyl)-2-(1H-imidazol-1-yl)-1-(benzotriazol-1-yl)ethyl]-1H-indole

In 20 mL of anhydrous tetrahydrofuran, introduce 0.3 g (0.81 mmol) of 2-(1H-benzotriazol-yl)-1-(4-fluorophenyl)-2-indol-1-ylethanol and 0.13 g (0.81 mmol) of 1,1′-carbonyldiimidazole. Stir and bring to reflux for 4 hours. Concentrate the solution and take up the residue in water. Extract with dichloromethane, dry the organic phase over anhydrous sodium sulfate and evaporate. Purify the evaporation residue by chromatography on silica gel with elution by a 19/1 dichloromethane/absolute ethanol mixture (Cpsé 83).

Yield: 64%

Melting point: 216-219° C. (diisopropyl ether)

### Example 47

### 1-[2-(4-Bromophenyl)-2-(1H-imidazol-1-yl)ethyl]-1H-indole

According to Example 10: Cpsé 84

Yield: 54%

Brown oil

TABLE 26
Final compounds according to example 10

| | |
|---|---|
| | (IE1) |
| 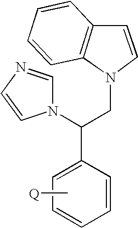 | |

| N^{o} Cpsé | Q | Derived from | Yld (%) | Mp (° C.) |
|---|---|---|---|---|
| 85 | 4-Cl | 104 | 40 | oil |
| 86 | 4-F | 106 | 35 | 61-63 |
| | | | | (dichloromethane) |

### Synthesis of Compounds of Formula (IG)

1. Preparation of the Intermediates

### Example 48

### Ethyl 1-methyl-1H-indazole-3-carboxylate and ethyl 2-methyl-2H-indazole-3-carboxylate

1a) Ethyl 1H-Indazole-3-carboxylate

In a hot solution of 4.1 g (102.5 mmol) of sodium hydroxide in 65 mL of water dissolve 14.7 g (100 mmol) of isatin. Cool this solution to 0° C. and add a solution at 0° C. of 6.9 g (100 mmol) of sodium nitrite in 25 mL of water. Add this reaction medium rapidly to a solution of 19.1 g (194.7 mmol) of concentrated sulfuric acid in 200 mL of water cooled to 0° C. by ice (the temperature of the reaction medium must not exceed 4° C. during the addition). At the end of the addition, stir for 15 minutes. Add to the reaction medium a solution of 54 g (239.3 mmol) of tin chloride dihydrate in 85 mL of concentrated hydrochloric acid cooled to 0° C. Leave to stir for 1 hour. Filter off the precipitate formed.

Heat to reflux for 2 hours a solution of 12 g of the recovered solid in 200 mL of ethanol in the presence of 6 g concentrated sulfuric acid. Evaporate the solvent and take up the oil obtained in dichloromethane. Wash the organic phase with a 4% solution of sodium bicarbonate then by a saturated sodium chloride solution. Dry the organic phase over anhydrous sodium sulfate, filter and evaporate to dryness. Purify the evaporation residue by successive passages through a silica gel column (dichloromethane/absolute ethanol 98/2; dichloromethane/diethyl ether: 80/20) (Int. 125).

Yield: 32%

Melting point: 135° C. (dichloromethane/absolute ethanol)

1b) Ethyl 1-methyl-1H-indazole-3-carboxylate and Ethyl 2-methyl-2H-indazole-3-carboxylate

In 12 mL of acetonitrile, place 1.71 g (5.28 mmol) of cesium carbonate and 0.5 g (2.63 mmol) of ethyl 1H-indazole-3-carboxylate. Heat to reflux for 2 hours. Add to the reaction medium 0.2 mL (3.2 mmol) of methyl iodide and continue the reflux for 30 minutes. Filter and wash the precipitate in dichloromethane Wash the organic phase in water, dry over anhydrous sodium sulfate and evaporate to dryness. Purify the evaporation residue by chromatography on silica gel with elution by a 70/30 petroleum ether/ethyl acetate mixture.

Ethyl 1-methyl-1H-indazole-3-carboxylate (Int. 126)

Yield: 39%

Melting point: 49° C. (petroleum ether/ethyl acetate)

Ethyl 2-methyl-2H-indazole-3-carboxylate (Int. 127)

Yield: 15%

Melting point: 60° C. (petroleum ether/ethyl acetate) 2. Preparation of the final compounds

### Example 49

### 3-(1H-Imidazol-1ylmethyl)-1-methyl-1H-indazole

According to Example 9: Cpsé 87

Yield: 55%

Colourless oil

### Synthesis of the Compounds of Formula (IH)

1. Preparation of the Intermediates

### Example 50

### 1-(4-Fluorobenzyl)-1H-7-azaindole-3-carboxaldehyde

1a) 1H-7-Azaindole-3-carboxaldehyde

To a solution of 1 g (8.46 mmol) of 1H-7-azaindole in 77 mL of a 1/1 nitromethane/1,2-dichloroethane mixture cooled to 0° C., add 3.9 mL (42.3 mmol) dichloromethyl methyl ether and 3.8 g (28.51 mmol) of aluminium chloride. Stir for 30 minutes and repeat twice with the same quantities of the chloro agent of aluminium chloride at 30 minute intervals. Add progressively 55 mL of water then 270 mL of a saturated sodium bicarbonate solution. Extract with diethyl ether, wash the organic phases with a saturated solution of sodium chloride, dry and evaporate to dryness (Int. 128).

Yield: 80%

Melting point: 204° C. (diethyl ether)

1b) 1-(4-Fluorobenzyl)-1H-7-azaindole-3-carboxaldehyde

In 20 mL of acetonitrile, introduce 1.46 g (10 mmol) of 1H-7-azaindole-3-carbaldehyde and 6.52 g (20 mmol) of cesium carbonate. Stir and bring to reflux for 2 hours. Add 1.59 g (11 mmol) of 4-fluorobenzyl chloride. Maintain the reflux for 1 hour. Filter the solution and evaporate the solvent. Take up the residue in water, extract with dichloromethane, dry the organic phase over anhydrous sodium sulfate and evaporate (Int. 129).

Yield: 92%

Yellow oil

2. Preparation of the Final Compound

### Example 51

### 1-(4-Fluorobenzyl)-3-(1H-Imidazol-1ylmethyl)-1H-7-azaindole

According to Example 10: Cpsé 88

Yield: 55%

Colourless oil

Pharmacological Activity of the Compounds of the Invention

The compounds of formula (I) possess antifungal and/or antiparasitic pharmacological properties.

It has been shown according to the invention that the compounds of formula (I) are endowed with antifungal activity. In addition, some have been shown to be effective against parasites of the genus Leishmania. The three clinical forms (cutaneous, cutaneo-mucosal and visceral) of leishmaniasis are very widespread in the third world and are on the increase in the developed countries since the appearance of AIDS.

### Example 52

### In Vitro Antifungal and Antiparasitic Activity of the Compounds of Formula (I) According to the Invention

A. Materials and Methods

A.1 Measurement of Antifungal Activity on a Clinical Strain of Candida albicans.

This protocol was adapted from the techniques described by Mosmann (1983), Levitz et al.(1985) and Pagé et al. (1993). This protocol has been restandardized in the Laboratory of Parasitology of the Faculty of Pharmacy of Nantes. The calorimetric test was performed on an Elisa microplate of 96 wells.

1—Preparation of the Solutions

The culture medium consisted of RPMI 1640 (100 mL), HEPES buffer (2 mL) and of glucose (2 g); the pH of the medium was 7. A mother solution 2 mM in the product to be tested was obtained by solubilizing the azole derivative in a solution of 2 mL of DMSO and 3 mL of physiological solution. A series of three solutions of the product to be tested, at concentrations of 100, 10 and 1 μM (or μg/mL), was obtained by successive dilutions.

A suspension of Candida albicans (strain n° CA980001) was also prepared, in a mixture of physiological solution/tween (0.01%). The counting of the Candida albicans by a Malassez cell enabled a dilution to be performed so as to place in each well 5.102 Candida per mL.

2—Filling the Wells of the Plate

In order to maintain adequate humidity for the test, the outside wells were filled with 200 μL of distilled water.

The blank of the test consisted of three wells, each well receiving only 100 μL of the culture medium. The control of the test also consisted of three wells, each well receiving 100 μL of the culture medium and 100 μL of the Candida albicans suspension.

The other wells were filled in the same way as the control, then 100 μL of each concentration of the solution to be tested was placed in 3 wells.

3—Incubation and Final Treatment

The plate was then incubated at 37° C. for 24 hours. At the end of the test, 10 μL of a solution of MTT* was added to each well and the plate was again placed in the incubator at 37° C. for 4 hours. The MTT was then reduced by a formazan dehydrogenase (colour: red-violet). The reaction was then stopped by addition of 100 μL of acidified isopropanol so as to solubilize the formazan and to neutralize the coloured indicator present in the RPMI. *3-(4,5-Dimethylthiazol-2-yl)-2,5-diphenyl tetrazolium bromide (pale yellow).

The results were read using a spectrophotometer, at a wavelength of 570 nm.

Under these experimental conditions, amphotericin B has a IC₉₀ or MIC (Minimum Inhibiting Concentration) of 0.12 μM (0.12 μg/mL). However, for fluconazole, the calculated MIC corresponds to a IC₈₀ of a value of 0.07 μM (0.02 μg/mL).

A.2. Measurement of the Antifungal Activity on a Clinical Strain of Aspergillus fumigatus.

This protocol was adapted from the techniques described by Mosmann (1983), Levitz et al.(1985) and Pagé et al. (1993). This protocol has been restandardized in the Laboratory of Parasitology of the Faculty of Pharmacy of Nantes. The colorimetric test was performed on an Elisa microplate of 96 wells.

1—Preparation of the Solutions

The culture medium consisted of RPMI 1640 (100 mL), HEPES buffer (2 mL) and of glucose (2 g); the pH of the medium was 7. A mother solution 2 mM in the product to be tested was obtained by solubilizing the azole derivative in a solution of 2 mL of DMSO and 3 mL of physiological solution. A series of three solutions of the product to be tested, at concentrations of 100, 10 and 1 μM (or μg/mL), was obtained by successive dilutions.

A suspension of spores of Aspergillus fumigatus (strain n°AF 980003) was also prepared, in a mixture of physiological solution/tween (0.01%). The counting of Aspergillus fumigatus by a Malassez cell enabled a dilution to be performed so as to place in each well 10⁴ par mL.

2—Filling the Wells of the Plate

In order to maintain adequate humidity for the test, the outside wells were filled with 200 μL of distilled water.

The blank of the test consisted of three wells, each well receiving only 100 μL of the culture medium. The control of the test also consisted of three wells, each well receiving 100 μL of the culture medium and 100 μL of the suspension of spores.

The other wells were filled in the same way as the control, then 100 μL of each concentration of the solution to be tested was placed in 3 wells.

3—Incubations and Final Treatment

The plate was incubated at 37° C. for 4 hours. The germination of the spores was monitored through a microscope. 150 μL of each concentration of the solution to be tested was placed in 3 wells and the plate was then incubated at 37° C. for 24 hours. At the end of the test, a solution of Alamar Blue®* (10 μL) was added to each well and the plate was again placed in the incubator at 37° C. for 20 hours. The solution of Alamar Blue® was thus reduced and the medium became pink in colour. *Alamar Blue@: fluorescence indicator (blue).

The results were read using a spectrofluorimeter, at wavelengths of 550 nm for the excitation and of 590 nm for the emission.

Under these experimental conditions, amphotericin B has a IC₉₀ or MIC (Minimum Inhibiting Concentration) of 0.15 μM (0.14 μg/mL).

A.3. Measurement of the Antiparasitic Activity on the Promastigote Form of Leishmania.

The evaluation of the in vitro activity on the promastigote stage of Leishmania mexicana was performed in a microplate of 96 wells, by calorimetric determination of the cell viability to MTT (Berman et al., 1980; Katiyar et al., 1992).

1—Preparation of the Solutions

The culture medium consisted of RPMI 1640 (100 mL), HEPES buffer (2 mL) and of glucose (2 g); the pH of the medium was 7. A mother solution 2 mM in the product to be tested was obtained by solubilizing the azole derivative in 5 mL of physiological serum. A series of three solutions of the product to be tested, at concentrations of 100, 10 and 1 μM (or μg/mL), was obtained by successive dilutions

A suspension (1 mL) of promastigotes (exponential phase) was also prepared at 2.106 promastigotes per mL.

2—Filling the Wells of the Plate

In order to maintain adequate humidity for the test, the outside wells were filled with 200 μL of distilled water.

The blank of the test consisted of three wells, each well receiving only 100 μL of the culture medium. The control of the test also consisted of three wells, each well receiving 100 μL of the culture medium and 100 μL of the promastigotes suspension.

The other wells were filled in the same way as the control, then 100 μL of each concentration of the solution to be tested was placed in 3 wells.

3—Incubation and Final Treatment

The plate was then incubated at 26° C. for 36 hours. At the end of the test, 10 μL of a solution of MTT was added to each well and the plate was again placed in the incubator at 26° C. for 4 hours. The MTT was then reduced by a mitochondrial formazan dehydrogenase (colour: red-violet). The reaction was then stopped by addition of 100 μL of acidified isopropanol so as to solubilize the formazan and to neutralize the coloured indicator present in the RPMI.

The results were read using a spectrophotometer, at a wavelength of 570 nm.

Under these experimental conditions, amphot ricin B has a IC₅₀ of 0.026 μM (0.029 μg/mL). However, for glucantime®, the calculated IC₅₀ is 15 000 μg/mL.

B. Results

The results are given in Tables 27 to 40.

The results for the compounds of formula (IA) are shown in Table 27.

The results for the compounds of formula (IB) and (IC) are shown in Tables 28 to 35.

The results for the compounds of formula (ID) are shown in Tables 36 to 38.

The results for the compounds of formula (IE) and (IF) are shown in Tables 39 and 40.

The results show that the compounds N° 29n, 31n, 40n, 43 and 66f have a significant activity of inhibition of Candida albicans. The most active compounds were compounds N° 48, 50, 51, 55n, 56n, 57, 58, 73, 74, 75, 76 and 77, these being significantly more active than the reference compounds AmB, Fluconazole and Glucantime.

The results show that compounds N°9, 31n, 50 and 57 were the most active against Aspergillus fumigatus, and that the compounds N°5, 6, 15, 33, 48, 58, 64n, 67f; 68f, 69n and 72n also have strong activity against this fungus.

The results show that compounds N° 31n, 32, 45, 47 and 50 were the most active against Leishmania mexicana and that the compounds N° 4, 7f, 9, 10, 33, 34 and 48 also have strong activity against this parasite.

### Example 53

### Antifungal Activity In Vitro on Several Clinical Strains of Candida.

A. Materials and Methods

Test In Vitro on Strains of Candida: albicans (4 strains), krusei (4 strains), lusitaniae (1 strain), parapsilosis (2 strains), tropicalis (1 strain).

This evaluation was performed in the same way as the in vitro tests carried out to determine the antifungal activity on a strain of Candida albicans CA980001 (Mosmann, 1983; Levitz et al., 1985; Pagé et al., 1993).

B. Results

The results are given in Table N°41.

The results show that the compound which was the most active against different strains of Candida was compound N°78 although compounds n°73 to 76 were also very active.

Compounds 73, 75 and 78 were active against strains of Candida albicans and one strain of Candida krusei.

Compounds 73 and 78 were very active against strains of Candida albicans and also against Candida krusei, Candida lusitaniae and Candida parapsilosis.

Compound 74 was very active against strains of Candida albicans, Candida lusitaniae and Candida parapsilosis.

Compound 76 was very active against strains of Candida albicans, and Candida lusitaniae.

### Example 54

### Antifungal Activity In Vivo on a Model of Disseminated Candidiasis

A. Materials and Methods

The anti-Candida albicans activity was evaluated in comparison with an untreated control and a reference treatment: either ketoconazole (intraperitoneal route) or fluconazole (oral route). According to techniques described by Abruzzo et al. (1997), Yotsuji et al. (1997) and Karyotakis et al. (1995).

The strain of Candida albicans (CA98001) was intravenously inoculated into female Swiss mice immunodeprived by hydrocortisone acetate (100 mg/kg). The treatment (5 days) was administered either by an intraperitoneal, or by oral route (in combination with 0.5% carboxymethylcellulose in distilled water) and began 2 hours after the inoculation.

The effectiveness of the treatment was evaluated over 14 days by 2 parameters: the survival and the contamination of certain organs (the kidney, spleen, liver and heart). After this time, the surviving mice were sacrificed and their organs cultured.

B. Results

### Test N°1:

At day 8 (D8), the survival of the animals treated with compound 75, at a daily dose of 20 mg/kg, was improved by 25% compared to the control batch.

Protocol used: compound 75 was administered at 20 mg/kg/d for 5 d and ketoconazole also at 20 mg/kg/d for 5 d.

Result: compound 75 had slight activity on the survival at D4-D5 but not on survival at the end of the experiment (D14).

For ketoconazole: 88% survival at D14.

### Test N°2

Antifungal Activity In Vivo of Compounds 75 and 78.

A model of invasive candidiasis was developed in female Swiss mice immunodeprived by subcutaneous injection of 100 mg/kg of hydrocortisone acetate. After inoculation of 5×10⁵ Candida albicans in the caudal vein, the treatment was performed intraperitoneally for 5 consecutive days at doses of 2×30 mg/kg/d for compounds 75 and 78 and of 10 mg/kg/d for the reference treatment (ketoconazole). A control batch injected with the vehicle was incorporated in the test. The animals were monitored for 14 days after the beginning of the treatment. The activity of the treatment is expressed by the percentage of mice which survived and its effectiveness by comparison with the vehicle control group.

This test showed that the treatment by compound 75 was ineffective in terms of the survival of the animals, thus confirming the in vivo results obtained before with this molecule in other therapeutic schemes. In contrast compound 78 was active and significantly prolonged the survival of the mice, from D9 to D14. In addition, only the treatment with the triazole derivative 78 gave a survival of 33% of the mice at D14.

The results are given in FIG. 1.

| | |
|---|---|
| Protocol used: | Compounds 75 and 78 administered at 2 × 30 mg/kg/d |
| | for 5 d. |
| | Control: Ketoconazole (10 mg/kg/d for 5 d). |
| Results: | Compound 75: no survival at end of experiment (D14). |
| | Compound 78: 33% survival at D14. |
| | Ketoconazole: no survival at D14. |

### Test N°3

Activity In Vivo of Compound 78 at 3×20 mg/kg/d (Intraperitoneal) in a Model of Systemic Murine Candidiasis.

The modification of the dosage to 3×20 mg/kg/d for 5 days considerably improved the survival of the mice compared to the untreated control group and to the group treated with ketoconazole. In fact 100% of the mice treated with compound 78 were alive at D14 while in the preceding tests a dosage of 2×30 mg/kg only gave a survival rate of 30% of the mice. In addition continuing the experiment for 4 more days showed a superiority of compound 78 over ketoconazole (20 mg/kg) by a survival 25% greater than that of the reference treatment.

Moreover the evaluation of the parasite level in the kidneys showed a significant reduction after treatment by compound 0.78 compared with the control group.

The results are shown on FIG. 2.

| | |
|---|---|
| Protocol used: | Compound 78 administered at 3 × 20 mg/kg/d for 5 d. |
| | Control: Ketoconazole (20 mg/kg/d for 5 d). |
| Results: | Compound 78: 100% survival at D14 and 75% |
| | survival at D18. |
| | Ketoconazole: 88% of survival at D14 and 50% |
| | survival at D18. |

### Test N°4

Activity In Vivo of Compound 78 at 3×20 mg/kg/d (Intraperitoneal and Oral) in a Model of Systemic Murine Candidiasis.

Protocol used: Compound 78 administered at 3×20 mg/kg/d for 5 d by intraperitoneal and oral routes.

Controls: Ketoconazole (20 mg/kg/d for 5 d and Fluconazole (5 mg/kg/d for 5 d).

Results:

Compound 78: 100% survival at D10 (Treatment intraperitoneal or per os).

Ketoconazole: 50% survival at D10.

Fluconazole: 100% survival at D10 (treatment per os).

### Example 55

### Anti-Leishmanian Activity on the Amastigote Form

A. Materials and Methods

The evaluation of the activity on the amastigote stage of Leishmania mexicana was performed in a microplate of 96 wells according to techniques described by Berman et al. (1980) and Katiyar et al. (1992).

1—Obtaining Peritoneal Macrophages of BALB/c Mice

An ip injection of RPMI to a BALB/c mouse was performed, followed by an abdominal massage. After sacrifice of the mouse, the injected solution was recovered in a sterile hemolysis tube. The cells were counted using a hematimeter. The solution was then centrifuged for 10 minutes, at 1500 r.p.m. After elimination of the supernatant, the residue was resuspended in a sufficient volume of RPMI 1640 to obtain a final concentration of 10⁶ macrophages/mL.

2—Adherence of the Macrophages to the Cell Culture Plate

Glass slides were then placed in each well of a 24-well plate. The suspended cells (106 macrophages/mL) were added and the plates were incubated at 37° C., with 5% of CO₂, for 12 hours.

3—Preparation of the Promastigotes

This test required the use of a culture of promastigotes of Leishmania mexicana in the stationary phase of growth. The cells were counted using a hematimeter. After dilution in RPMI 1640, a concentration of 2.10⁶ promastigotes was obtained.

4—Infestation of the Macrophages and Sensitization to the Molecules

After elimination of the culture medium, each well was washed twice with RPMI 1640. The parasites in suspension were then added and each plate was incubated at 37° C. with 5% of CO₂ for 24 hours. Each well was washed once with RPMI 1640. The solution of each concentration of the substance to be tested (100, 10 and 1 μM (or μg/mL)), prepared with RPMI 1640, was added to the well and the plate was incubated at 37° C. with 5% of CO₂ for 96 hours. The culture medium was changed every 2 days. After fixation of the material adhered to the slides by methanol for 10 minutes, the coloration to May-Grunwald-Giemsa and reading with a microscope were performed.

Under these experimental conditions, amphotericin B has a IC₅₀ of 0.47 μg/mL. In contrast, for meglumine antimoniate, the calculated IC₅₀ was 48.7 μg/mL.

5—Calculation of the Percentage Inhibition at 1 μM on Intracellular Amastigotes

For the most active molecules which had been evaluated by the test on the peritoneal macrophages infected by the intracellular amastigote form of Leishmania mexicana, it was possible to determine a percentage inhibition. For the parasite level corresponding to 1 μM, the number of amastigotes per macrophage was calculated and then compared with the control to obtain a percentage inhibition.

B. Results

The results are given in Table 42.

The results show that compounds N° 45 and 47 are very active against the intracellular amastigote form of Leishmania.

These compounds thus show a very strong antiparasitic activity under conditions very close to those in which they are likely to act in vivo, inside the cells.

### Example 56

### Anti-Leishmanian Activity In Vivo on a Model of Cutaneo-Mucosal and Visceral Leishmaniasis

A. Materials and Methods

The anti-Leishmania activity was evaluated by comparison with an untreated control and a reference treatment (meglumine antimoniate, Glucantime® at a dose of 10 mg/Kg/d) according to techniques described by Lepape et al. (1999), Croft et al. (1993) and Hill et al. (1983).

The animals used (Centre d'élevage R. Janvier, Le Genest-St—France) were male BALB/c mice, aged 7 weeks at the time of inoculation of the parasites. This operation was performed in accordance with the legislation relating to animal experimentation.

The administration of the metacyclic promastigotes (2×10⁶ in 100 μL), derived from Leishmania major, was performed by the subcutaneous route, on the posterior pad of the left paw.

Days after the inoculation, the treatment was administered intraperitoneally for 10 days. Three weeks after the treatment, its effectiveness was determined by determining the parasite level in the liver, spleen, popliteal ganglion and the cutaneous lesion.

B. Results

Under these conditions, the administration of compound 45 caused a strong reduction of the visceral parasite level. It reached 79% for the hepatic level and 98.9% for the spleen level. Glucantime® in contrast, reduced the hepatic level by 74% and spleen level by 82%.

### Example 57

### Study of the Mechanism of Action of Compound 78

A. Materials and Methods

The principal mechanism of action of the triazole derivatives is the inhibition of the synthesis of ergosterol leading to instability of the cell membrane. In order to demonstrate this mechanism of action for compound 78, we used two techniques to quantify the ergosterol present in a sample, one method of high-performance liquid chromatography (Standard NF V18-112) and one method of direct reading by UV spectrophotometry described by Arthington-Skaggs et al. (1999). These two techniques require a prior saponification of the sample followed by an extraction of the sterols present. The results obtained are expressed in μg of ergosterol per g of sample. The activity of the derivatives studied is expressed by their capacity to inhibit the synthesis of ergosterol compared to an untreated control sample.

B. Results

The preliminary results showed a strong inhibition of the synthesis of ergosterol by compound 78. The HPLC determination in fact showed a reduction of 70% of the presence of ergosterol after treatment for 24 hours at 10 μg. Under the same treatment conditions, the second technique of UV spectrophotometry showed an absence of ergosterol in the treated sample.

TABLE 27
Compounds of formula (IA)

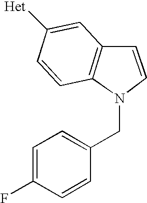

| | | CMI or CI₈₀ | | | | CI₅₀ | |
|---|---|---|---|---|---|---|---|
| | | Candida albicans | | Aspergillus fumigatus | | Leishmania mexicana | |
| | | (CA980001) | | (AF980003) | | Promastigotes | |
| N^{o} Cpsé | Het | μM | μg/ml | μM | μg/ml | μM | μg/ml |
| 1 | Imid | 49 ± 10 | 14 ± 3 | | | | |
| 2 | 1-Tetrazol | 70 ± 2 | 20 ± 1 | | | | |
| AmB* | | 0.12 | 0.12 | 0.15 ± 0.04 | 0.14 ± 0.04 | 0.026 ± 0.02 | 0.029 ± 0.03 |
| Fluconazole | | 0.07 | 0.02 | — | — | — | — |
| Glucantime ® | | — | — | — | — | — | 15000 ± 260 |

*AmB: CMI = CI₉₀ for Candida albicans and Aspergillus fumigatus.

TABLE 28
Compounds of formulas (IB) and (IC)
Sub-series with straight chain

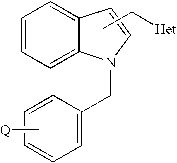

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| | Site of | | | (CA980001) | | (AF980003) | | Promastigotes | |
| N^{o} Cpsé | fixation | Q | Het | μM | μg/ml | μM | μg/ml | μM | μg/ml |

| | | | | | |
|---|---|---|---|---|---|
| 3n* | 2 | 4-F | Imid | 16 ± 6 | 6 ± 2 |

| | | | | | | |
|---|---|---|---|---|---|---|
| AmB** | 0.12 | 0.12 | 0.15 ± 0.04 | 0.14 ± 0.04 | 0.026 ± 0.02 | 0.029 ± 0.03 |
| Fluconazole | 0.07 | 0.02 | — | — | — | — |
| Glucantime ® | — | — | — | — | — | 15000 ± 260 |

*n = nitrate.
**AmB: CMI = CI₉₀ for Candida albicans and Aspergillus fumigatus.

TABLE 29
Compounds of formulas (IB) and (IC)

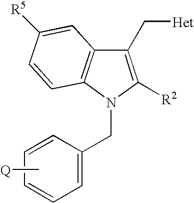

| | | | | | CMI or CI₈₀ | | | | CI₅₀ | |
|---|---|---|---|---|---|---|---|---|---|---|
| | | | | | Candida albicans | | Aspergillus fumigatus | | Leishmania mexicana | |
| | | | | | (CA980001) | | (AF980003) | | Promastigotes | |
| N^{o} Cpsé | R² | R⁵ | Q | Het | μM | μg/ml | μM | μg/ml | μM | μg/ml |
| 4 | H | H | 2-F | Imid | 64 ± 1 | 19.54 ± 1 | >100 | — | 10 ± 1.5 | 3.0 ± 0.5 |
| 5 | H | H | 3-F | Imid | 68 ± 1 | 20.76 ± 1 | 46 ± 5 | 14 ± 1.5 | 30.4 ± 2.5 | 9.2 ± 0.7 |
| 6 | H | H | 2.4-diF | Imid | 48 ± 4 | 16 ± 2 | 60 ± 2 | 19.4 ± 2 | 18.5 ± 1.8 | 6 ± 0.5 |
| 7f* | H | H | tert-Bu | Imid | >100 | — | 70 ± 3 | 32.17 ± 3 | 8.4 ± 0.5 | 3.9 ± 0.09 |
| 9 | H | Br | 2-Cl | Imid | 81 ± 14 | 32.46 ± 14 | 21 ± 0.3 | 8 ± 1.2 | 8.5 ± 0.2 | 3.4 ± 0.09 |
| 10 | H | Br | 4-Cl | Imid | >100 | — | >100 | — | 7.5 ± 1.2 | 3 ± 0.5 |
| 11 | H | Br | 2-F | Imid | >100 | — | >100 | — | 47.7 ± 1.6 | 18.3 ± 0.6 |
| 12 | H | Br | 4-F | Imid | >100 | — | >100 | — | 20.6 ± 2.4 | 7.9 ± 0.9 |
| 13 | H | Br | | | 59 ± 5 | 23 ± 2 | | | | |
| 14 | H | Cl | 4-Cl | Imid | 85 | 30 | 71 ± 3 | 25 ± 1 | non linear | — |
| 15 | H | F | 4-Cl | Imid | 21 ± 1 | 7 ± 1 | 61 ± 3 | 20.73 ± 3 | 15.9 ± 5 | 5.4 ± 1.6 |

| | | | | | | |
|---|---|---|---|---|---|---|
| AmB** | 0.12 | 0.12 | 0.15 ± 0.04 | 0.14 ± 0.4 | 0.026 ± 0.02 | 0.029 ± 0.03 |
| Fluconazole | 0.07 | 0.02 | — | — | — | — |
| Glucantime ® | — | — | — | — | — | 15000 ± 260 |

*f = fumarate.
**AmB: CMI = CI₉₀ for Candida albicans and Aspergillus fumigatus.

TABLE 30
Compounds of formulas (IB) and (IC)

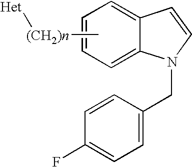

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| | Site of | | | (CA980001) | | (AF980003) | | Promastigotes | |
| N^{o} Cpsé | fixation | Het | n | μM | μg/mL | μM | μg/mL | μM | μg/mL |
| 17 | 4 | Imid | 1 | 54 ± 1 | 16.49 ± 1 | 72 ± 3 | 21.99 ± 2 | | |
| 18 | 5 | Imid | 1 | 58 ± 4 | 18 ± 1 | | | | |
| 19n* | 6 | Imid | 1 | 77 ± 1 | 28.36 ± 1 | >100 | — | | |

| | | | | | | |
|---|---|---|---|---|---|---|
| AmB** | 0.12 | 0.12 | 0.15 ± 0.04 | 0.14 ± 0.04 | 0.026 ± 0.02 | 0.029 ± 0.03 |
| Fluconazole | 0.07 | 0.02 | — | — | — | — |
| Glucantime ® | — | — | — | — | — | 15000 ± 260 |

*n = nitrate.
**AmB: CMI = CI₉₀ . for Candida albicans and Aspergillus fumigatus

TABLE 31
Compounds of formulas (IB) and (IC)

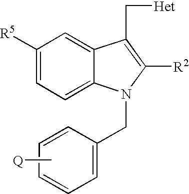

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| N° | | | | (CA980001) | | (AF980003) | | Promastigotes | |
| Cpsè | R⁵ | Q | Het | μM | μg/mL | μM | μg/mL | μM | μg/mL |
| 20 | H | 2-Cl | 1-Triazol | >100 | — | >100 | — | 65 | 21 |
| 21 | H | 2-Cl | 4-Triazol | >100 | — | >100 | — | 25 + 3.3 | 8.07 ± 1 |
| 22 | Br | 2-Cl | 1-Triazol | >100 | — | >100 | — | 59.2 | 23.8 |
| 23 | Br | 4-Cl | 1-Triazol | >100 | — | >100 | — | >100 | — |
| 24 | Br | 2-F | 1-Triazol | >100 | — | >100 | — | 79.8 | 30.74 |
| 25 | Br | 4-F | 1-Triazol | >100 | — | >100 | — | 50.3 | 19.38 |
| AmB* | | | | 0.12 | 0.12 | 0.15 ± 0.04 | 0.14 ± 0.04 | 0.026 ± 0.02 | 0.029 ± 0.03 |
| Fluconazole | | | | 0.07 | 0.02 | — | — | — | — |
| Glucantime ® | | | | — | — | — | — | — | 15000 ± 260 |

*AmB:CMI = CI₉₀ (red framing) for Candida albicans and Aspergillus fumigatus.

TABLE 32
Compounds of formulas (IB) and (IC)
Sub-series with branched chain

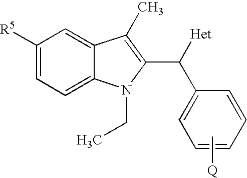

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| N° | | | | (CA980001) | | (AF980003) | | Promastigotes | |
| Cpsè | R⁵ | Q | Het | μM | μg/mL | μM | μg/mL | μM | μg/mL |
| 26n* | H | 3-Br | Imid | 60 ± 1 | 27 ± 1 | | | | |
| 27n* | H | 4-Br | Imid | 57 ± 4 | 25 ± 2 | | | | |
| 28n* | H | 3-Cl | Imid | 58 ± 2 | 24 ± 1 | | | | |
| 29n* | H | 4-Cl | Imid | 6 ± 1 | 2 ± 1 | | | | |
| 30 | H | 2,4-diCl | Imid | 76 ± 2 | 25 ± 1 | | | | |
| 31n* | H | 4-F | Imid | 7 ± 0.3 | 2.77 ± 0.3 | 16 ± 3 | 6 ± 1 | <0.1 | |
| 32 | Br | 3-Cl | Imid | 62 ± 1 | 27 ± 0.5 | 66 ± 5 | 28 ± 2 | <0.1 | |
| 33 | Br | 4-Cl | Imid | 59 ± 1 | 25 ± 0.5 | 56 ± 3 | 24 ± 1 | 6.7 ± 2.1 | 2.87 ± 2 |
| 34 | Br | 2,4-diCl | Imid | 48 ± 0.5 | 22 ± 0.5 | >100 | — | 4.28 ± 0.1 | 1.99 ± 0.04 |
| AmB** | | | | 0.12 | 0.12 | 0.15 ± 0.04 | 0.14 ± 0.04 | 0.026 ± 0.02 | 0.029 ± 0.03 |
| Fluconazole | | | | 0.07 | 0.02 | — | — | — | — |
| Glucantime ® | | | | — | — | — | — | — | 15000 ± 260 |

*n = nitrate.
**AmB:CMI = CI₉₀ for Candida albicans and Aspergillus fumigatus.

TABLE 33
Compounds of formulas (IB) and (IC)

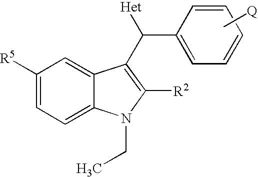

| | | | | | CMI or CI₈₀ | | | | CI₅₀ | |
|---|---|---|---|---|---|---|---|---|---|---|
| | | | | | Candida albicans | | Aspergillus fumigatus | | Leishmania mexicana | |
| N° | | | | | (CA980001) | | (AF980003) | | Promastigotes | |
| Cpsè | R⁵ | R² | Q | Het | μM | μg/mL | μM | μg/mL | μM | μg/mL |
| 40n* | H | H | 3-Cl | Imid | 7 ± 1 | 3.1 ± 1 | | | | |
| 41n* | H | H | 4-Cl | Imid | 19 ± 10 | 8 ± 4 | | | | |
| 42n* | H | H | 2,4-diCl | Imid | 73 ± 3 | 32 ± 1 | | | | |
| 43 | H | CH₃ | 4-F | Imid | 6 ± 1 | 2.0 ± 1 | 53 ± 3 | 17 ± 1 | 5.9 ± 0.14 | 1.88 ± 0.1 |
| 45 | Br | H | 2,4-diCl | Imid | >100 | — | >100 | — | 0.23 ± 0.04 | 0.1 ± 0.01 |
| 46 | Br | H | 4-F | Imid | 66 ± 6 | 26 ± 2 | >100 | — | 35 ± 3 | 14 ± 1 |
| 47 | Br | H | 2,4-diCl | 1-Triazol | >100 | — | >100 | — | 0.37 ± 0.1 | 0.16 ± 0.04 |
| AmB** | | | | | 0.12 | 0.12 | 0.15 ± 0.04 | 0.14 ± 0.04 | 0.026 ± 0.02 | 0.029 ± 0.03 |
| Fluconazole | | | | | 0.07 | 0.02 | — | — | — | — |
| Glucantime ® | | | | | — | — | — | — | — | 15000 ± 260 |

*n = nitrate.
**AmB: CMI = CI₉₀ for Candida albicans and Aspergillus fumigatus.

TABLE 34
Compounds of formulas (IB) and (IC)

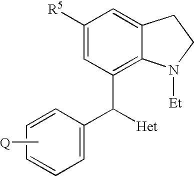

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| N° | | | | (CA980001) | | (AF980003) | | Promastigotes | |
| Cpsè | R⁵ | Q | Het | μM | μg/mL | μM | μg/mL | μM | μg/mL |
| 48 | H | 3-Cl | Imid | 0.06 ± 0.01 | 0.02 ± 0.01 | 58 ± 4 | 26 ± 2 | 3 ± 1 | 1 ± 0.3 |
| 50 | H | 4-F | Imid | 0.67 ± 0.01 | 0.22 ± 0.01 | 5.4 ± 0.2 | 1.7 ± 0.1 | 0.74 ± 0.017 | 0.24 ± 0.017 |
| 51 | Br | 4-F | Imid | 0.7 ± 0.01 | 0.28 ± 0.01 | >100 | — | 16.6 ± 3.33 | 6.64 ± 3.33 |
| AmB* | | | | 0.12 | 0.12 | 0.15 ± 0.04 | 0.14 ± 0.04 | 0.026 ± 0.02 | 0.029 ± 0.03 |
| Fluconazole | | | | 0.07 | 0.02 | — | — | — | — |
| Glucantime ® | | | | — | — | — | — | — | 15000 ± 260 |

*AmB:CMI = CI₉₀ (red framing) for Candida albicans and Aspergillus fumigatus.

TABLE 35
Compounds of formulas (IB) and (IC)

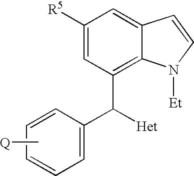

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| N° | | | | (CA980001) | | (AF980003) | | Promastigotes | |
| Cpsè | R⁵ | Q | Het | μM | μg/mL | μM | μg/mL | μM | μg/mL |
| 55n* | H | 3-Cl | Imid | 0.098 | 0.039 | >100 | — | | |
| 56n* | H | 4-Cl | Imid | 0.4 | 0.16 | | | | |
| 57 | H | 4-F | Imid | 0.67 ± 0.02 | 0.21 ± 0.01 | 20 ± 5 | 6 ± 2 | 2.49 ± 1.3 | 0.80 ± 1.3 |
| 58 | Br | 4-F | Imid | 0.69 ± 0.01 | 0.27 ± 0.01 | 59 ± 4 | 23 ± 2 | 6.2 ± 0.35 | 2.4 ± 0.12 |
| AmB** | | | | 0.12 | 0.12 | 0.15 ± 0.04 | 0.14 ± 0.04 | 0.026 ± 0.02 | 0.029 ± 0.03 |
| Fluconazole | | | | 0.07 | 0.02 | — | — | — | — |
| Glucantime ® | | | | — | — | — | — | — | 15000 ± 260 |

*n = nitrate.
**AmB:CMI = CI₉₀ for Candida albicans and Aspergillus fumigatus.

TABLE 36
Compounds of formula (ID)

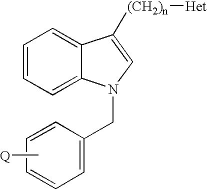

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| N° | | | | (CA980001) | | (AF980003) | | Promastigotes | |
| Cpsè | Q | Het | n | μM | μg/mL | μM | μg/mL | μM | μg/mL |
| 59 | 4-Cl | Imid | 2 | >100 | — | >100 | — | 87 | 15.6 |
| 60 | 4-Cl | Imid | 3 | >100 | — | >100 | — | 74.7 | 26.1 |
| 61n* | 4-Cl | Imid | 4 | >100 | — | >100 | — | 62 ± 0.3 | 20.05 ± 0.3 |
| AmB** | | | | 0.12 | 0.12 | 0.15 ± 0.04 | 0.14 ± 0.04 | 0.026 ± 0.02 | 0.029 ± 0.03 |
| Fluconazole | | | | 0.07 | 0.02 | — | — | — | — |
| Glucantime ® | | | | — | — | — | — | — | 15000 ± 260 |

*n = nitrate.
**AmB:CMI = CI₉₀ for Candida albicans and Aspergillus fumigatus.

TABLE 37
Compounds of formula (ID)

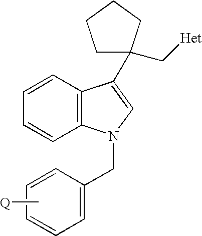

| | | | CMI or CI₈₀ | | | | CI₅₀ | |
|---|---|---|---|---|---|---|---|---|
| | | | Candida albicans | | Aspergillus fumigatus | | Leishmania mexicana | |
| N° | | | (CA980001) | | (AF980003) | | Promastigotes | |
| Cpsè | Q | Het | μM | μg/mL | μM | μg/mL | μM | μg/mL |
| 62 | 2-Cl | CH | >100 | — | >100 | — | 72.8 | 28.38 |
| 63 | 4-Cl | CH | >100 | — | >100 | — | 14.3 ± 3.9 | 5.5 ± 1.5 |
| AmB* | | | 0.12 | 0.12 | 0.15 ± 0.04 | 0.14 ± 0.04 | 0.026 ± 0.02 | 0.029 ± 0.03 |
| Fluconazole | | | 0.07 | 0.02 | — | — | — | — |
| Glucantime ® | | | — | — | — | — | — | 15000 ± 260 |

*AmB:CMI = CI₉₀ for Candida albicans and Aspergillus fumigatus.

TABLE 38
Compounds of formula (ID)

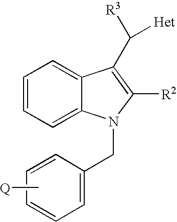

| | | | | | CMI or CI₈₀ | | | | CI₅₀ | |
|---|---|---|---|---|---|---|---|---|---|---|
| | | | | | Candida albicans | | Aspergillus fumigatus | | Leishmania mexicana | |
| N° | | | | | (CA980001) | | (AF980003) | | Promastigotes | |
| Cpsè | R² | Q | Het | R³ | μM | μg/mL | μM | μg/mL | μM | μg/mL |
| 64n* | H | 4-Cl | Imid | ethyl | 36 ± 10 | 15 ± 4 | 57 ± 5 | 23.53 ± | 7.2 ± 0.07 | 2.97 ± 0.02 |
| | | | | | | | | 5 | | |
| 65f** | H | 4-Cl | Imid | n-propyl | >100 | — | >100 | — | 78.7 ± 9.5 | 37.7 ± 4.5 |
| 66f** | H | 4-Cl | Imid | n-butyl | 7 ± 0.1 | 3.46 ± | >100 | — | 5.4 ± 0.06 | 2.6 ± 0.03 |
| | | | | | | 0.1 | | | | |
| 67f** | H | 4-F | Imid | methyl | 57 ± 2 | 25 ± 1 | 64 ± 2 | 28 ± 1 | 3.6 ± 1.2 | 1.57 ± 1.2 |
| 68f** | H | 2,4- | Imid | methyl | 31 ± 7 | 15 ± 3 | 65 ± 3 | 31 ± 1 | 5.7 ± 0.13 | 2.7 ± 0.05 |
| | | diCl | | | | | | | | |
| 69n* | H | 2,4- | Imid | methyl | 65 ± 6 | 26 ± 5 | 64 ± 5 | 21.59 ± | | |
| | | diF | | | | | | 5 | | |
| 71n* | CH₃ | 2,4- | Imid | methyl | 52 ± 3 | 23.31 ± | 74 ± 3 | 28.44 ± | | |
| | | diCl | | | | | | 1 | | |
| 72 | CH₃ | 4-F | Imid | methyl | 67 ± 3 | 22 ± 1 | 69 ± 1 | 23 ± 1 | 2 ± 1 | 0.7 ± 0.3 |
| AmB*** | | | | | 0.12 | 0.12 | 0.15 ± 0.04 | 0.14 ± 0.04 | 0.026 ± 0.02 | 0.029 ± 0.03 |
| Fluconazole | | | | | 0.07 | 0.02 | — | — | — | — |
| Glucantime ® | | | | | — | — | — | — | — | 15000 ± 260 |

*n = nitrate.
**f = fumarate.
***AmB:CMI = CI₉₀ for Candida albicans and Aspergillus fumigatusp.

TABLE 39
Compounds of formulas (IE) and (IF)

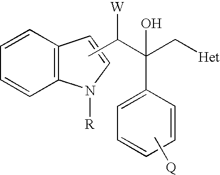

| | | | | | | CMI or CI₈₀ | | | | CI₅₀ | |
|---|---|---|---|---|---|---|---|---|---|---|---|
| | | | | | | Candida albicans | | Aspergillus fumigatus | | Leishmania mexicana | |
| | Site of | | | | | (CA980001) | | (AF980003) | | Promastigotes | |
| N° | fixation | R | W | Q | Het | μM | μg/ml | μM | μg/ml | μM | μg/ml |
| 73 | 1 | — | H | 4-Br | Imid | 0.06 ± | 0.02 ± | >100 | — | 6.65 ± 0.22 | 2.7 ± 0.08 |
| | | | | | | 0.001 | 0.001 | | | | |
| 74 | 1 | — | H | 4-Cl | Imid | 0.07 ± | 0.02 ± | 83 ± 4 | 29 ± 1 | 15.05 ± 0.6 | 5 ± 0.2 |
| | | | | | | 0.003 | 0.001 | | | | |
| 75 | 1 | — | H | 2,4-diCl | Imid | <0.001 | | 4 ± 0 | 1 ± 0 | 4 | 1.47 |
| 76 | 1 | — | H | 4-F | Imid | 0.08 ± | 0.03 ± | 88 ± 2 | 30 ± 0.7 | 9.2 ± 0.09 | 3 ± 0.03 |
| | | | | | | 0.002 | 0.001 | | | | |
| 77 | 1 | — | H | 2,4-diF | Imid | 0.0011 | 0.00035 | 11 ± 7 | 4 ± 2 | 4.5 | 1.59 |
| 78 | 1 | — | H | 2,4-diCl | Triaz | <0.001 | | | | <1 | |
| 79 | 1 | — | H | 4-CF₃ | Triaz | <0.001 | | | | | |
| 80 | 1 | — | CH₃ | 2,4-diCl | Triaz | | | | | | |
| 81 | 1 | — | CH₃ | 2,4-diF | Triaz | <0.001 | | | | | |
| 82 | 3 | tosyl | H | 2,4-diCl | Triaz | | | | | | |
| AmB* | | | | | | 0.12 | 0.12 | 0.15 ± 0.04 | 0.14 ± 0.04 | 0.026 ± 0.02 | 0.029 ± 0.03 |
| Fluconazole | | | | | | 0.07 | 0.02 | — | — | — | — |
| Itraconazole | | | | | | — | — | 0.71 | 0.5 | — | — |
| Ketoconazole | | | | | | 0.0094 | 0.005 | | | | 3 ± 0.5 |
| Antimoine | | | | | | — | — | — | — | — | 4300 ± 50 |
| Glucantime ® | | | | | | — | — | — | — | — | 15000 ± 260 |

*AmB:CMI = CI₉₀ for Candida albicans and Aspergillus fumigatus.

TABLE 40
Compounds of formulas(IE) and (IF)

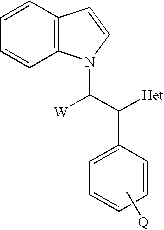

| | | | | CMI or CI₈₀ | | | | CI₅₀ | |
|---|---|---|---|---|---|---|---|---|---|
| | | | | Candida albicans | | Aspergillus fumigatus | | Leishmania mexicana | |
| N° | | | | (CA980001) | | (AF980003) | | Promastigotes | |
| Cpsè | Q | Het | W | μM | μg/mL | μM | μg/mL | μM | μg/mL |
| 83 | 4-F | Imid | Benzotriazole | 55 ± 5 | 23 ± 2 | >100 | — | 4.4 ± 0.07 | 1.85 ± 0.03 |
| 84 | 4-Br | Imid | H | >100 | — | 68 ± 3 | 29 ± 1 | 8.45 ± 1.02 | 3.6 ± 0.4 |
| 85 | 4-Cl | Imid | H | >100 | — | 88 ± 5 | 28 ± 2 | >100 | — |
| AmB** | | | | 0.12 | 0.12 | 0.15 ± 0.04 | 0.14 ± 0.04 | 0.026 ± 0.02 | 0.029 ± 0.03 |
| Fluconazole | | | | 0.07 | 0.02 | — | — | — | — |
| Glucantime ® | | | | — | — | — | — | — | 15000 ± 260 |

*n = nitrate.
**AmB:CMI = CI₉₀ for Candida albicans and Aspergillus fumigatus

TABLE 41
CI₈₀ (μM) on clinical strains of Candida:
C. albicans (4), C. krusei (4), C. lusitaniae (1),
C. parapsilosis (2), C. tropicalis (1):

| Strain | Cpsé73 | Cpsé74 | Cpsé75 | Cpsé76 | Cpsé78 | Ketoconazole | AmB |
|---|---|---|---|---|---|---|---|
| CA980001 | 0.06 ± 0.002 | 0.061 ± 0.004 | <0.001 | 0.067 ± 0.005 | <0.001 | 0.005 | 0.08 ± 0.001 |
| CA980002 | 0.63 ± 0.016 | 0.47 ± 0.017 | 0.65 ± 0.01 | 0.61 ± 0.022 | <0.01 | | 0.07 ± 0.001 |
| CA980003 | 0.55 ± 0.0963 | 0.07 ± 0.0047 | 0.64 ± 0.01 | 0.25 ± 0.11 | <0.01 | | 0.08 ± 0.002 |
| CA980005 | 0.53 ± 0.022 | 0.3 ± 0.024 | 0.71 ± 0.01 | 0.44 ± 0.044 | <0.01 | | 0.08 ± 0.002 |
| CK980001 | 0.65 ± 0.01 | 59.29 ± 1.33 | 0.62 ± 0.002 | 65.38 ± 3.71 | <0.01 | | 0.07 ± 0.01 |
| CK980002 | 12.81 ± 2.22 | 7.74 ± 0.13 | ND | 38.05 ± 7.24 | 7.2 ± 0.1 | 0.7 ± 0.02 | 0.15 ± 0.08 |
| CK980003 | 92.27 ± 3.90 | >100 | >1 | 85.27 ± 0.324 | 9.2 ± 0.04 | 0.8 ± 0.02 | 0.86 ± 0.0 |
| CK980004 | >100 | >100 | 4.2 ± 0.1 | >100 | 0.3 ± 0.1 | <0.01 | 0.84 ± 0.0 |
| CL980001 | 0.63 ± 0.01 | 0.63 ± 0.01 | 0.3 ± 0.1 | 0.69 ± 0.08 | <0.01 | <0.01 | 0.63 ± 0.01 |
| CP980001 | 0.67 ± 0.0 | 0.69 ± 0.03 | 1.0 ± 0.1 | 4.10 ± 0.35 | <0.01 | | >1 |
| CP980002 | 4.65 ± 0.11 | 3.71 ± 1.246 | 0.86 ± 0.04 | 6.32 ± 0.14 | 0.53 ± 0.12 | <0.01 | 0.81 ± 0.01 |
| CT980002 | — | — | ND | — | <0.01 | | 0.08 ± 0.0 |

CA = Candida albicans; CK = Candida krusei; CL = Candida lusitaniae; CP = Candida parapsilosis; CT = Candida tropicalis.

TABLE 42
Anti-leishmanian activity on the amastigote form

| | CI₅₀ (μg/mL) | |
|---|---|---|
| Molecules | Promastigotes | Intracellular amastigotes |
| Meglumine antimoniate | 4300 ± 50 | 48.7 ± 8.4 |
| Ketoconazole | 3 ± 0.5 | 1.3 ± 0.2 |
| AmB | 0.029 ± 0.03 | 0.47 ± 0.05 |
| 45 | 0.1 ± 0.01 | 1.5 ± 0.01 |
| 47 | 0.16 ± 0.04 | 3.1 ± 0.04 |

### REFERENCES

Abruzzo G. K., Flattery et al. Gill C. J., Kong L., Smith J. G., Pikounis V. B., Balkovec J. M., Bouffard A. F., Dropinski J. F., Rosen H., Kropp H., Bartizal K., Evaluation of the Echinocandin Antifungal MK-0991 (L-743,872): Efficacies in Mouse Models of Disseminated Aspergillosis, Candidiasis, and Cryptococcosis, Antimicrob. Agents Chemother., 1997, 41, 2333-2338.

Arthington-Skaggs, B. A., Jrari, H., Desai, T., Morrison, C. J., Quantitation of Ergosterol Content: Novel Method for Determination of Fluconazole Susceptibility of Candida albicans, J. Clin. Microbiol., 1999, 37, 3332-3337.

Andriole et al., 1999, J. Antimicrobial Chemotherapy, 44: 151-162.

Berman J. D., Wyler D., An in Vitro Model for Investigation of Chemotherapeutic Agents in Leishmaniasis, J. Infect. Dis., 1980, 142, 83-86.

Cavrini V. et al., 1984, Arch. Pharm., 317 :662-668

Croft S. L., Neal R. A., Herrmann D. J., Antileishmania Activity of the Ether Phospholipid Ilmofosine, Trans Roy. Soc. Trop. Med. Hyg., 1993, 87, 217-219.

Croft, S. L., Neal, R. A., Herrmann, D. J., Antileishmania Activity of the Ether Phospholipid Ilmofosine, Trans Roy. Soc. Trop. Med. Hyg., 1993, 87, 217-219.

Gatti R. et al., Arch. Pharm., 1985, 318, 157-160.

Georgopapadakou N. H. et al., Microb. Agents Chemother. 1996 40(2): 279-291.

Hill, J. O., North, R. J., Collins F. M., Advantages of measuring changes in the number of viable parasites in murine models of experimental cutaneous leishmaniasis, Infect. Immun., 1983, 39,1087-1094.

Joly V. et al., 1994, Antif ngiques: données générales, in <<Médicaments antiinfectieux>>, Médecine Scienhces, Cirban C., Régnier B., Saimot G., Vildé J. L., Yeni P., Flammarion, France.

Katiyar J. C., et al. Anuradha, Sharma S., Kala-Azar: Current Status of Experimental Chemotherapy, Med. Res. Rev., 1992, 12, 473-504.

Karyotakis, N. C. et al., Dignani M. C., Anaissie E. J., SCH 51048, a New Antifungal Triazole Active against Hematogenous Candida krusei Infections in Neutropenic Mice, Antimicrob. Agents Chemother., 1995, 39, 775-777.

Le Borgne M. et al., Arch. Pharm. Pharm. Med. Chem., 1997, 330,141-145.

Le Borgne M. et al., Bioorg., 1999, & Med. Chem. Lett., 9, 333-336.

Lepape, P., Abdala H. et al., Pagniez F., Robert J. M., Le Baut G, Activity of N-lutidinylarylcarboxamides against Leishmania donovani and L. braziliensis, Acta Parasitologica, 1999, 44,156-159.

Lepape, P., Abdala, H., Pagniez, F., Robert, J-M., Le Baut, G., Activity of N-lutidinylarylcarboxamides against Leishmania donovani and L. braziliensis, Acta Parasitologica, 1999, 44, 156-159.

Levitz S. M., R. D. et al. Diamond, A Rapid Colorimetric Assay of Fungal Viability with the Tetrazolium Salt MTT, J. Inf. Dis., 1985, 152, 938-945.

Marchand P. et al., Pharm. Pharmacol. Commun. 1998, 4, 211-218

Mosmann T., Rapid Colorimetric Assay for Cellular Growth and Survival: Application to Proliferation and Cytotoxicity Assays, J. Immunol. Methods, 1983, 65, 55-63.

Mosmann, T Rapid Colorimetric Assay for Cellular Growth and Survival: Application to Proliferation and Cytotoxicity Assays, J. Immunol. Methods, 1983, 65, 55-63.

Pagé B., Pagé M, Noël., C. et al., A New Fluorimetric Assay for Cytotoxicity Measurements in vitro, Int. J. Oncol., 1993, 3, 473-476.

Yotsuji, A., Shimizu K., Araki H., Fujimaki K., Nishida N., Hori R., Annen N., Yamamoto S., Hayakawa H., Imaizumi H., Watanabe Y., Narita H., T-8581, a New Orally and Parenterally Active Triazole Antifungal Agent: In Vitro and In Vivo Evaluations, Antimicrob. Agents Chemother., 1997, 41, 30-34.

1. Antifungal and/or antiparasitic pharmaceutical composition comprising, by way of active principle, a compound of formula (I) below: 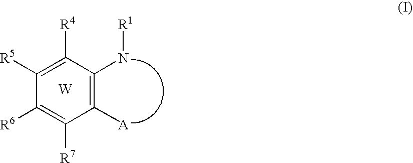
in which:
A represents a bivalent radical chosen from among the following radicals:
(a) CR²═CR^{3,},
(b) CHR²—CHR³ or
(c) N═CR³, N being bound to the nitrogen atom of the NR¹ group represented in formula (I)
at least one of the radicals R¹ to R⁷ represents the linkage (CRR′)ₘ—(CR″R′″)ₙ—(CHX)ₚ-Het in which:
a)—R and R′ independently from each other represent hydrogen, a lower alkyl, alkenyl, cycloalkyl, phenyl, substituted phenyl, halogenophenylalkyl or benzotriazolyl group; or
R and R′ together form a saturated ring with five or six members, unsubstituted of substituted by a lower alkyl group or a halogen chosen from bromine, chlorine or fluorine;
b) R″ and R′″ independently from each other represent a lower alkyl, phenyl, substituted phenyl, halogenophenylalkyl, hydroxy, alkoxy or acyloxy group;
c) Het represents an 1H-imidazol-1-yl, 2-methyl-1H-imidazol-1-yl, 1H-1,2,4-triazol-1-yl, 4H-1,2,4-triazol-4-yl or 1H-tetrazol-5-yl group;
d) X represents a hydrogen or a lower alkyl, phenyl or substituted phenyl group;
e) m, n and p are independently from each other equal to 0, 1, 2, 3, 4 or 5;
the other radicals R¹ to R⁷ which are not part of the (CRR′)ₘ—(CR″R′″)ₙ—(CHX)ₚ-Het linkage independently from each other represent a hydrogen atom, a lower alkyl group, a halogen, a lower trifluoroalkyl, cyano, alkoxy, alkoxycarbonyl, carboxamido, phenyl, substituted phenyl, phenylalkyl or halogenophenylalkyl group.
the ring 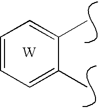
represents
either the phenyl nucleus, the central unit corresponding in this case to indole, indoline, indazole,
or the pyridine nucleus, the nitrogen being located in position 4, 5, 6 or 7 of the central bicyclic ring corresponding in this case to azaindole.
On condition that:
(1) when A represents N═CR³, the radical R¹ is different from a substituted benzyl radical or a substituted or unsubstituted ethyl radical and the radical R³ represents a hydrogen atom; and
(2) when A represents CR²═CR³, the radical R¹ is different from a substituted or unsubstituted benzyl radical and the radicals R² and R³ both represent a hydrogen atom.
(3) when A represents CR²═CR³ and the radical R³ represents 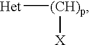
Het is different from a 1H-imidazol-1-yl ring,
or an enantiomer or a diastereoisomer of the compound of formula (I) or a salt from the addition to an acid of a compound of formula (I), in combination with a pharmaceutically acceptable vehicle.

2. Pharmaceutical composition according to claim 1, characterized in that it comprises a compound of formula (IA) below: 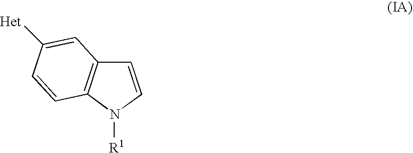
in which:
R¹ represents hydrogen or an alkyl, phenylalkyl, or substituted phenylalkyl group, and
Het represents an 1H-imidazol-1-yl, 1H-1,2,4-triazol-1-yl or 1H-tetrazol-5-yl group.

3. Pharmaceutical composition according to claim 2, characterized in that the compound of formula (IA) is 1-(4-fluorobenzyl)-5-(1H-imidazol-1-yl)-1H-indole.

4. Pharmaceutical composition according to claim 2, characterized in that the compound of formula (IA) is 1-(4-fluorobenzyl)-5-(1H-tetrazol-5-yl)-1H-indole.

5. Pharmaceutical composition according to claim 1, characterized in that it comprises a compound of formula (IB) below: 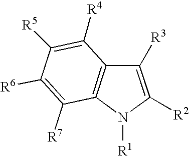
in which:
at least one of the radicals R² to R⁷ represents a group 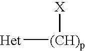
in which:
X represents hydrogen, alkyl, phenyl, halogenophenyl and Het represents a 1H-imidazol-1-yl, 1H-1,2,4-triazol-1-yl or 4H-1,2,4-triazol-4-yl ring and p is equal to 0, 1, 2, 3, 4 or 5, with the exception of the compounds of formula (IB) in which R represents 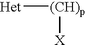
with Het representing a 1H-imidazol-1-yl ring,
the R² to R⁷ radicals not forming part of the group 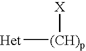
independently from each other represent a group chosen from among hydrogen, alkyl, alkoxy, alkoxycarbonyl, halogenoalkyl or cyano;
the radical R¹ represents hydrogen, or a phenyl, substituted phenyl, phenylalkyl or halogenophenylalkyl group.

6. Pharmaceutical composition according to claim 5, characterized in that the compound of formula (IB) is chosen from among the following compounds:
1-(2-Chlorobenzyl)-3-(1H-1,2,4-triazol-1-ylmethyl)-1H-indole
1-(2-Chlorobenzyl)-3-(4H-1,2,4-triazol-4-ylmethyl)-1H-indole.

7. Composition according to claim 5, characterized in that the compound is of formula (IB1) below: 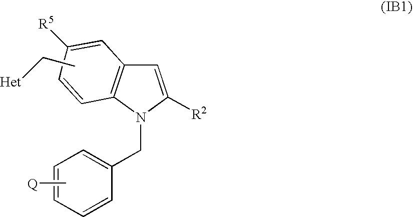
in which
R⁵ represents hydrogen, bromine, chlorine, fluorine or the methoxy group, R² represents hydrogen or the methyl group and Het represents the imidazolyl group bonded in the position 3, 4, 5 or 6 or the 1-triazolyl group bonded in position 3.

8. Composition according to claim 5, characterized in that the compound is of formula (IB2) below: 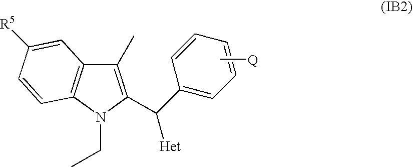
in which:
R⁵ represents hydrogen or bromine, Het represents an imidazolyl or 1-triazolyl group and Q represents one or two atoms of bromine or chlorine bonded to the positions 2, 3 or 4.

9. Composition according to claim 5, characterized in that the compound is of formula (IB3) below: 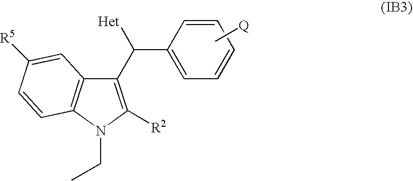
in which:
R² represents hydrogen or the methyl group, R⁵ represents hydrogen or a bromine atom, Het represents the imidazolyl group and Q represents one or two chlorine or fluorine atoms bonded to the positions 2, 3 or 4.

10. Composition according to claim 5, characterized in that the compound is of formula (IB4) below: 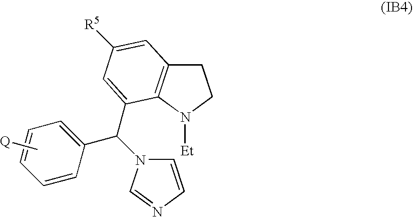
in which:
R⁵ is hydrogen or a chlorine atom, Et represents the ethyl radical and Q represents a chlorine or fluorine atom bonded in position 3 or 4.

11. Composition according to claim 5, characterized in that the compound is of formula (IB5) below: 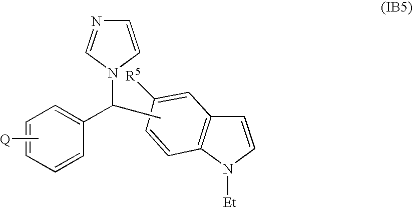
in which:
R⁵ represents hydrogen or bromine, Et represents the ethyl radical and Q represents a bromine, chlorine, or fluorine atom bonded in position 3 or 4.

12. Pharmaceutical composition according to claim 1, characterized in that it comprises a compound of formula (IC) below: 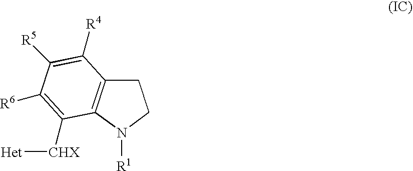
in which:
X represents hydrogen, alkyl, phenyl, halogenophenyl and Het represents an 1H-imidazol-1-yl, 1H-1,2,4-triazol-1-yl or 4H-1,2,4-triazol-4-yl ring;
the radicals R⁴ to R⁷ independently from each other represent a group chosen from among hydrogen, alkyl, alkoxy, alkoxycarbonyl, halogenoalkyl or cyano;
the radical R¹ represents hydrogen, or a phenyl, substituted phenyl, phenylalkyl or halogenophenylalkyl group.

13. Pharmaceutical composition according to claim 5, characterized in that the compound is chosen from among the following compounds:
1-Ethyl-2-[(4-fluorophenyl)(1H-imidazol-1-yl)methyl]-3-methyl-1H-indole;
5-Bromo-1-ethyl-2-[(4-fluorophenyl)(1H-1,2,4-triazol-1-yl)methyl]-3-methyl-1H-indole
5-Bromo-1-ethyl-3-[(4-fluorophenyl)(1H-imidazol-1yl)methyl]-1H-indole;
5-Bromo-1-ethyl-7-[(4-fluorophenyl)(1H-imidazol-1-yl)methyl]indoline;
5-[(4-Chlorophenyl)(1H-imidazol-1-yl)methyl]-1-ethyl-1H-indole.

14. Pharmaceutical composition according to claim 1, characterized in that it comprises a compound of formula (ID) below: 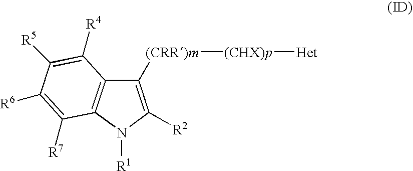
in which:
R³ represents the linkage (CRR′)ₘ—(CHX)ₚ-Het in which:
a)—R and R′ independently from each other represent hydrogen, a lower alkyl, alkenyl, cycloalkyl, phenyl, substituted phenyl, halogenophenylalkyl or benzotriazolyl group; or
R and R′ together form a five- or six-membered saturated ring, either unsubstituted or substituted by a lower alkyl group or a halogen chosen from among bromine, chlorine and fluorine.
c) Het represents an 1H-imidazol-1-yl, 2-methyl-1H-imidazol-1-yl, 1H-1,2,4-triazol-1-yl, 4H-1,2,4-triazol-4-yl or tetrazol-5-yl group;
d) X represents a hydrogen or a lower alkyl, phenyl or halogenophenyl group;
d) m and p are independently from each other equal to 0, 1, 2, 3, 4 or 5;
the radicals R¹, R², R⁴, R⁵, R⁶ and R⁷ represent, independently from each other, a hydrogen atom, a lower alkyl group, a halogen, a lower trifluoroalkyl, cyano, alkoxy, alkoxycarbonyl, carboxamido, phenyl, substituted phenyl, phenylalkyl or halogenophenylalkyl group.

15. Pharmaceutical composition according to claim 9, characterized in that the compound of formula (ID) is chosen from among the following compounds:
1-(4-Chlorobenzyl)-3-(2-1H-imidazol-1-ylethyl)-1H-indole;
1-[1-(4-Chlorobenzyl)-1H-indol-3-yl]-1-(1H-imidazol-1-ylmethyl)-cyclopentane;
1-(2,4-Dichlorobenzyl)-3-[(1H-imidazol-1-yl)(methyl)methyl]-2-methyl-1H-indole.

16. Pharmaceutical composition according to claim 14, characterized in that the compound is of formula (ID1) below: 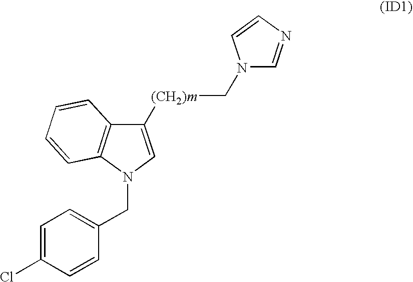
in which m is equal to 1, 2, 3, 4 or 5.

17. Pharmaceutical composition according to claim 14, characterized in that the compound is of formula (ID2) below:

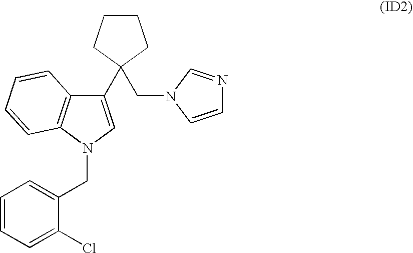

18. Pharmaceutical composition according to claim 14, characterized in that the compound is of formula (ID3) below: 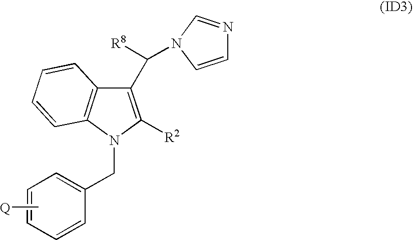
in which:
R⁸ represents an ethyl, n-butyl or methyl group; and
Q represents one or two atoms of chlorine, bromine or fluorine and R is as defined for formula (ID).

19. Pharmaceutical composition according to claim 1, characterized in that it comprises a compound of formula (IE) below: 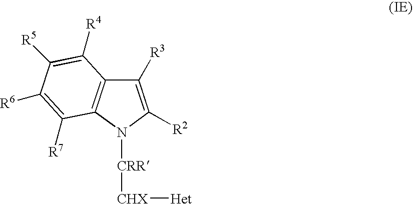
in which:
a)—R and R′ represent independently from each other hydrogen, a lower alkyl, alkenyl, cycloalkyl, phenyl, substituted phenyl, halogenophenylalkyl or benzotriazolyl group; or
R and R′ together form a five- or six-membered saturated ring, either unsubstituted or substituted by a lower alkyl group or a halogen chosen from among bromine, chlorine or fluorine;
b) Het represents a 1H-imidazol-1-yl, 2-methyl-1H-imidazol-1-yl, 1H-1,2,4-triazol-1-yl, 4H-1,2,4-triazol-4-yl or tetrazol-5-yl group;
c) X represents a hydrogen or a lower alkyl, phenyl or halogenophenyl group;
the radicals R² to R⁷ represent, independently from each other, a hydrogen atom, a lower alkyl group, a halogen, a lower trifluoroalkyl, cyano, alkoxy, alkoxycarbonyl, carboxamido, phenyl, substituted phenyl, phenylalkyl or halogenophenylalkyl group.

20. Pharmaceutical composition according to claim 19, characterized in that the compound is of formula (IE1) below: 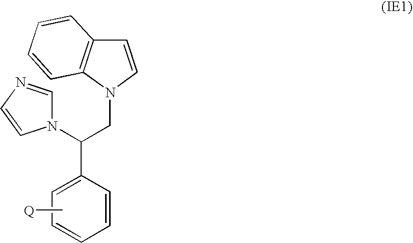
in which Q represents an atom of chlorine or of fluorine bonded to position 4.

21. Pharmaceutical composition according to claim 19, characterized in that the compound is 1-[2-(4-fluorophenyl)-2-(1H-imidazol-1-yl)-1-(benzotriazol-1-yl)ethyl]-1H-indole.

22. Pharmaceutical composition according to claim 1, characterized in that the compound is of formula (IF), below: 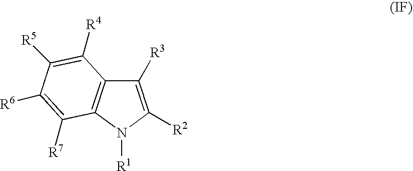
in which:
at least one of the radicals R¹ to R⁷ represents the linkage
Het-(CHX)—(CR″R′″)—(CRR′)ₘ
in which:
a) R and R′ represent independently from each other hydrogen or an alkyl group,
b) R″ represents a phenyl or substituted phenyl group,
c) R′″ represents hydrogen or a hydroxy, alkoxy or acyloxy group,
d) Het represents a 1H-imidazol-1-yl, 2-methyl-1H-imidazol-1-yl, 1H-1,2,4-triazol-1-yl, 4H-1,2,4-triazol-4-yl or tetrazol-5-yl group;
e) m is equal to 1 or 2;
f) X represents a hydrogen or a lower alkyl, phenyl or halogenophenyl group;
the other radicals R¹ to R⁷ which are not part of the linkage
Het-(CHX)—(CR″R′″)—(CRR′)ₘ
represent, independently from each other, a hydrogen atom, a lower alkyl, alkoxy, halogenoalkyl, or cyano group or a halogen atom.

23. Composition according to claim 22, characterized in that the compound is of formula.(IF1) below: 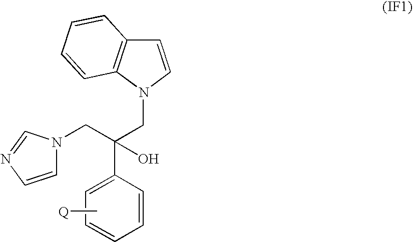
in which Q represents one or two atoms of chlorine or of fluorine bonded to positions 2 and/or 4.

24. Pharmaceutical composition according to claim 22, characterized in that the compound is 2-(4-bromophenyl)-1-(1H-imidazol-1-yl)-3-(indol-1-yl)propan-2-ol.

25. Pharmaceutical composition according to claim 1, characterized in that the compound is of formula (IG) below: 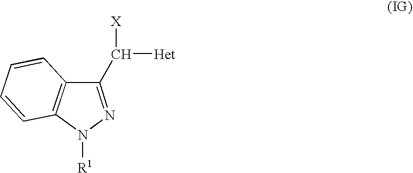
in which:
R¹ represents hydrogen, a substituted phenyl, phenylalkyl or substituted phenylalkyl group;
Het represents a 1H-imidazol-1-yl or 1H-1,2,4-triazol-1-yl radical;
X represents hydrogen, or an alkyl, phenyl or substituted phenyl group.

26. Pharmaceutical composition according to claim 25, characterized in that the compound of formula (IG) is 3-(1H-imidazol-1ylmethyl)-1-methyl-1H-indazole.

27. Pharmaceutical composition according to claim 1, characterized in that it comprises a compound of formula (IH) below: 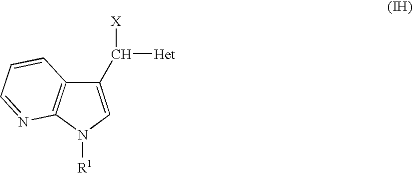
in which:
R¹ represents hydrogen, a substituted phenyl, phenylalkyl or substituted phenylalkyl group;
Het represents a 1H-imidazol-1-yl or 1H-1,2,4-triazol-1-yl radical;
X represents hydrogen, or an alkyl, phenyl or substituted phenyl group.

28. Pharmaceutical composition according to claim 27, characterized in that the compound is 1-(4-fluorobenzyl)-3-(1H-Imidazol-1ylmethyl)-1H-7-azaindole.

29. Pharmaceutical composition according to one of claims 1 to 28, characterized in that it is in a form suitable for oral administration.

30. Pharmaceutical composition according to one of claims 1 to 28, characterized in that it is in a form suitable for topical administration.

31. Pharmaceutical composition according to one of claims 1 to 28, characterized in that it is in a form suitable for parenteral or intravenous administration.

32. As novel compounds, the compounds of formula (I) as defined in claim 1, as well as their enantiomers and their diastereoisomers and their addition salts with acids.

33. As novel compounds, the compounds chosen from among the following compounds:
the compounds of formula (IA) such as defined in claim 2;
the compounds of formula (IB) such as defined in claim 5;
the compounds of formula (IB1) such as defined in claim 7;
the compounds of formula (IB2) such as defined in claim 8;
the compounds of formula (IB3) such as defined in claim 9;
the compounds of formula (IB4) such as defined in claim 10;
the compounds of formula (IB5) such as defined in claim 11;
the compounds of formula (IC) such as defined in claim 12;
the compounds of formula (ID) such as defined in claim 14;
the compounds of formula (ID1) such as defined in claim 16;
the compounds of formula (ID2) such as defined in claim 17;
the compounds of formula (ID3) such as defined in claim 18;
the compounds of formula (IE) such as defined in claim 19;
the compounds of formula (IE1) such as defined in claim 20;
the compounds of formula (IF) such as defined in claim 22.
the compounds of formula (IF1) such as defined in claim 23;
the compounds of formula (IG) such as defined in claim 25; et
the compounds of formula (IH) such as defined in claim 27.

34. Compound according to claim 33, characterized in that it is 1-(4-fluorobenzyl)-5-(1H-imidazol-1-yl)-1H-indole

35. Compound according to claim 33, characterized in that it is 1-(4-fluorobenzyl)-5-(1H-tetrazol-5-yl)-1H-indole.

36. Compound according to claim 33, characterized in that it is chosen from among the following compounds:
1-(4-Fluorobenzyl)-2-(1H-imidazol-1-ylmethyl)-1H-indole;
5-Bromo-1-(4-Chlorobenzyl)-3-(1H-imidazol-1-ylmethyl)-1H-indole;
1-(2-Chlorobenzyl)-3-(1H-1,2,4-triazol-1-ylmethyl)-1H-indole;
1-(2-Chlorobenzyl)-3-(4H-1,2,4-triazol-4-ylmethyl)-1H-indole.

37. Compound according to claim 33, characterized in that it is chosen from among the following compounds:
1-Ethyl-2-[(4-fluorophenyl)(1H-imidazol-1-yl)methyl]-3-methyl-1H-indole;
5-Bromo-1-ethyl-2-[(4-fluorophenyl)(1H-1,2,4-triazol-1-yl)methyl]-3-methyl-1H-indole
5-Bromo-1-ethyl-3-[(4-fluorophenyl)(1H-imidazol-1yl)methyl]-1H-indole;
5-Bromo-1-ethyl-7-[(4-fluorophenyl)(1H-imidazol-1-yl)methyl]indoline; and
5-[(4-Chlorophenyl)(1H-imidazol-1-yl)methyl]-1-ethyl-1H-indole.

38. Compound according to claim 33, characterized in that it is chosen from among the following compounds:
1-(4-Chlorobenzyl)-3-[(2-(1H-imidazol-1-yl)ethyl)]-1H-indole;
1-[1-(4-Chlorobenzyl)-1H-indol-3yl]-1-(1H-imidazol-1-ylmethyl)-cyclopentane;
1-(2,4-dichlorobenzyl)-3-[(1H-imidazol-1-yl)(methyl)methyl]-2-methyl-1H-indole.

39. Compound according to claim 33, characterized in that it is chosen from among the following compounds:
2-(4-Bromophenyl)-1-(1H-imidazol-1-yl)-3-(indol-1-yl)propan-2-ol; and
1-[2-(4-Fluorophenyl)-2-(1H-imidazol-1-yl)-1-(benzotriazol-1-yl)ethyl]-1H-indole.

40. Compound according to claim 33, characterized in that it is 3-(1H-Imidazol-1ylmethyl)-1-methyl-1 H-indazole;

41. Compound according to claim 33, characterized in that it is 1-(4-Fluorobenzyl)-3-(1H-imidazol-1ylmethyl)-1H-7-azaindole.

42. Use of a compound according to any of claims 32 to 41 for the production of an antifungal and/or antiparasitic pharmaceutical composition.

43. Use according to claim 42, characterized in that the pharmaceutical composition is in a form suitable for oral administration.

44. Use according to claim 42, characterized in that the pharmaceutical composition is in a form suitable for topical administration.

45. Use according to claim 42, characterized in that the pharmaceutical composition is in a form suitable for parenteral or intravenous administration.
